SEQUENCE LISTING (29 sequences)

SEQ ID NO: 1 - protein, 37 aa, artificial sequence
  Synthetic Construct
FAEKLKEAVKDYFAKLWDPAAEKLKEAVKDYFAKLWD

SEQ ID NO: 2 - protein, 37 aa, artificial sequence
  synthetic construct
FAEKFKEAVKDYFAKFWDPAAEKLKEAVKDYFAKLWD

SEQ ID NO: 3 - protein, 37 aa, artificial sequence
  synthetic construct
FAEKLKEAVKDYFAKLWDPFAEKLKEAVKDYFAKLWD

SEQ ID NO: 4 - protein, 37 aa, artificial sequence
  synthetic construct
FAEKFKEAVKDYFAKFWDPFAEKFKEAVKDYFAKFWD

SEQ ID NO: 5 - protein, 37 aa, artificial sequence
  synthetic construct
FAEKFKEAVKDYFAKFWDGAAEKLKEAVKDYFAKLWD

SEQ ID NO: 6 - protein, 37 aa, artificial sequence
  synthetic construct
FAEKFKEAVKPYFAKFWDPAAEKLKEAVKPYFAKLWD

SEQ ID NO: 7 - protein, 38 aa, artificial sequence
  synthetic construct
CFAEKFKEAVKDYFAKFWDPAAEKLKEAVKDYFAKLWD

SEQ ID NO: 8 - protein, 36 aa, artificial sequence
  synthetic construct
FAEKFKEAVKDYFAKFWDPAAEKLKEAVKDYFAKLW

SEQ ID NO: 9 - protein, 43 aa, artificial sequence
  synthetic construct
HHHHHHFAEKFKEAVKDYFAKFWDPAAEKLKEAVKDYFAKLWD

SEQ ID NO: 10 - protein, 37 aa, artificial sequence
  synthetic construct
  MOD_RES (1)..(1): acetylation
  MOD_RES (37)..(37): NH2
DWLKAFYDKVAEKLKEAAPDWFKAFYDKVAEKFKEAF

SEQ ID NO: 11 - protein, 37 aa, artificial sequence; identical to SEQ ID NO: 10
  synthetic construct

SEQ ID NO: 12 - protein, 37 aa, artificial sequence; identical to SEQ ID NO: 2
  synthetic construct
  MOD_RES (1)..(1): fluorescein amidite

SEQ ID NO: 13 - protein, 37 aa, artificial sequence; identical to SEQ ID NO: 2
  synthetic construct
  MOD_RES (1)..(1): biotinylation

SEQ ID NO: 14 - protein, 37 aa, artificial sequence; identical to SEQ ID NO: 2
  synthetic construct
  MOD_RES (37)..(37): pyrenebutyric acid

SEQ ID NO: 15 - protein, 37 aa, artificial sequence
  synthetic construct
FAEKFKEAVKDYFAKLWDPAAEKLKEAVKDYFAKLWD

SEQ ID NO: 16 - protein, 37 aa, artificial sequence
  synthetic construct
FAEKFKEAVKDYFAKFWDPAAEKLKEAVKDYFAKFWD

SEQ ID NO: 17 - protein, 37 aa, artificial sequence
  synthetic construct
FAEKFKEAVKDYFAKFWDPAAEKFKEAVKDYFAKLFD

SEQ ID NO: 18 - protein, 37 aa, artificial sequence
  synthetic construct
FAEKLKEAVKDYLAKFWDPAAEKLKEAVKDYFAKLWD

SEQ ID NO: 19 - protein, 37 aa, artificial sequence
  synthetic construct
FADKFKDAVKEYFAKFWEPAADKLKDAVKEYFAKLWE

SEQ ID NO: 20 - protein, 37 aa, artificial sequence
  synthetic construct
FAERFREAVRDYFARFWDPAAERLREAVRDYFARLWD

SEQ ID NO: 21 - protein, 37 aa, artificial sequence
  synthetic construct
WAEKFKEAVKDYWAKFWDPAAEKLKEAVKDYWAKLWD

SEQ ID NO: 22 - protein, 37 aa, artificial sequence
  synthetic construct
YAEKFKEAVKDYFAKFYDPAAEKLKEAVKDYFAKLWD

SEQ ID NO: 23 - protein, 37 aa, artificial sequence
  synthetic construct
FVEKFKEVAKDYFVKFWDPVVEKLKEVAKDYFVKLWD

SEQ ID NO: 24 - protein, 37 aa, artificial sequence
  synthetic construct
FAEKFKEAVKDQFAKFWDPAAEKLKEAVKDQFAKLWD

SEQ ID NO: 25 - protein, 37 aa, artificial sequence
  synthetic construct
FAEAFKEAVKDYFAKFWDPAAEKLKEAVKDYFAKLWD

SEQ ID NO: 26 - protein, 37 aa, artificial sequence
  synthetic construct
FAEAFKEAVKDYFAAFWDPAAEKLKEAVKDYFAKLWD

SEQ ID NO: 27 - protein, 37 aa, artificial sequence
  synthetic construct
FAEAFKEAVKDYFAAFWDPAAEKLKEAVADYFAKLWD

SEQ ID NO: 28 - protein, 37 aa, artificial sequence
  synthetic construct
FAAKFKEAVKDYFAKFWDPAAEKLAEAVADYFAALWD

SEQ ID NO: 29 - protein, 37 aa, artificial sequence; identical to SEQ ID NO: 5
  synthetic construct

(19) United States
(12) Patent Application Publication
(10) Pub. No.: US 2021/0253643 A1
(43) Pub. Date: Aug. 19, 2021

(54) HYDROPHOBIC BIOMOLECULE STABILIZING SCAFFOLD PEPTIDES AND METHODS OF MAKING AND USING SAME

(71) Applicant: The University of British Columbia, Vancouver (CA)

(72) Inventors: Franck DUONG, Vancouver (CA); Michael CARLSON, Vancouver (CA); Harveer DHUPAR, Vancouver (CA)

(21) Appl. No.: 17/059,854

(22) PCT Filed: Jun. 6, 2019
(86) PCT No.: PCT/CA2019/050792

§ 371 Date: Nov. 30, 2020

Related U.S. Application Data
(60) Provisional application No. 62/681,212, filed on Jun. 6, 2018.

(51) Int. Cl. C07K 14/00; C07K 1/14

(52) U.S. Cl. CPC C07K 14/001; C07K 1/145

(57) ABSTRACT

Scaffold peptides and methods of making and using same. The scaffold peptides have a general structure from the N-terminus to the C-terminus, AH₁-linker-AH₂. AH₁ and AH₂ each comprise an alpha helical structure and may fold against each other about the linker to form a bi-helical structure. The scaffold peptide includes alternating polar and non-polar regions. The non-polar regions of the scaffold peptide interact with the hydrophobic regions of the proteins to solubilize same in aqueous solutions without the presence of detergent. In some embodiments, the scaffold peptides are used for hydrophobic protein purification, isolation and/or stabilization.

### REFERENCE TO RELATED APPLICATIONS

This application claims priority to, and the benefit of, U.S. provisional patent application No. 62/681,212 filed 6 Jun. 2018, the entirety of which is incorporated by reference herein for all purposes.

### TECHNICAL FIELD

Some embodiments of the present invention relate to the field of hydrophobic biomolecule purification, isolation and/or stabilization. Some embodiments of the present invention relate to the field of hydrophobic protein purification, isolation and/or stabilization. Some embodiments of the present invention relate to the field of peptides capable of solubilizing hydrophobic proteins for the purification thereof without the use of detergent, and methods of making and using same. Some embodiments of the present invention relate to complexes containing hydrophobic proteins stabilized by peptides. In some aspects, the hydrophobic proteins are membrane proteins.

### BACKGROUND

Hydrophobic biomolecules such as membrane proteins perform essential biological functions, such as membrane transport, signal transduction, cell homeostasis, and energy metabolism. Despite their importance, obtaining these proteins in a stable non-aggregated state for research remains problematic. Membrane proteins are generally purified in detergent micelles. Detergents are often detrimental to protein structure and activity. They are also known to interfere with downstream analytical methods.

Detergent-free alternatives to membrane protein purification have been developed. However, known detergent-free membrane protein purification systems are laborious, costly and difficult to perform. One example is the use of scaffold proteins such as the nanodisc system, described in e.g. U.S. Pat. No. 7,592,008. The nanodisc system involves the use of two amphipathic membrane scaffold proteins (MSPs) apolipoprotein A1 (apoA-1) for wrapping around a small patch of lipid bilayer containing the target membrane protein (T. H. Bayburt, Y. V. Grinkova, S. G. Sligar, Arch. Bio-chem. Biophys. 2006, 450 (2), 215-222.; I. G. Denisov, Y. V. Grinkova, A. A. Lazarides, S. G. Sligar, J. Am. Chem. Soc. 2004, 126 (11), 3477-3487; I. G. Denisov, S. G. Sligar, Nat. Struct. Mol. Biol. 2016, 23 (6), 481-486.). The formation of a nanodisc depends on many factors such as lipid to protein ratio, scaffold length, nature of lipids, rate of detergent removal and overall amenability of the target for re-assembly into a lipid bilayer (Bayburt et al., 2006; Denisov et al., 2004; F. Hagn, M. Etzkorn, T. Raschle, G. Wagner, J. Am. Chem. Soc. 2013, 135 (5), 1919-1925). Small deviations from optimal conditions result in liposome formation or protein aggregation, which can lead to low-efficiency reconstitution (Bayburt et al., 2006).

Peptides have been considered as an alternative to scaffold proteins. Examples include peptergents (K. Corin, P. Baaske, D. B. Ravel, J. Song, E. Brown, X. Wang, C. J. Wienken, M. Jerabek-Willemsen, S. Dufur, Y. Luo, D. Braun, S. Zhang. PLos One, 2011, 6(11): e25067), lipopeptides (H. Tao, S. C. Lee, A. Moeller, R. T. Roy, F. Y. Siu, J. Zimmerman, R. C. Stevens, C. S. Potter, B. Carragher and Q. Zhang. Nat. Methods. 2013, 10, 759-761.), nanostructured [beta]-sheet peptides (G. G. Privé, Curr. Opin. Struct. Biol., 2009, 379-385.), and beltides (A. N. Larsen, K. K. Sørensen, N. T. Johansen, A. Martel, J. J. K. Kirkensgaard, K. J. Jensen, L. Arleth, S. R. Midtgaard, Soft Matter 2016, 12 (27), 5937-5949.). Peptergents and lipopeptides can solubilize membrane proteins directly from a lipid bilayer, but these peptides form mixed micelles that readily aggregate and precipitate below a certain critical micellar concentration (Corin et al., 2011; Tao et al., 2013). Beltides were shown to trap bacteriorhodopsin in solution, but the method required prior incubation with specific amounts of lipids and the particles formed have been reported to be unstable at physiological temperatures (Larsen et al., 2016). Cost and complexity of the peptide can also be problematic. Nanostructured [beta]-sheet peptides contain extended alkyl chains covalently linked to glycine residues, while lipopeptides need to be covalently linked to a lipid molecule (Tao et al., 2013; Privé, 2009). These hydrophobic peptides are also difficult to work with given their low solubility. Peptergents require titration of base to become soluble in aqueous solution (Corin et al., 2011), and [beta]-sheet peptides can form extended filament clusters without detergent (Privé, 2009). Thus, a peptide-based membrane protein reconstitution method that is cost-effective, rapid, unhindered by issues of solubility and generally applicable to membrane proteins remains to be developed.

There remains a need for improved hydrophobic biomolecule purification methods and for improved tools for purifying and studying hydrophobic biomolecules. There remains a need for simple, rapid and cost-effective methods to stabilize hydrophobic proteins without detergent, and in particular, a need for scaffold peptides capable of stabilizing any hydrophobic proteins without having to optimize the purification conditions for each hydrophobic protein of interest.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Aspects of this invention relate to an amphipathic scaffold peptide. The amphipathic scaffold peptide may have an amino acid sequence having the general formula, from the N-terminus to the C-terminus, AH₁-linker-AH₂. AH₁ and AH₂ may each include at least one aromatic amino acid residue. In some embodiments, the number of aromatic amino acid residues in AH₁ may be greater than the number of aromatic amino acid residues in AH₂. In some embodiments, the number of aromatic amino acid residues in AH₁ is equal to the number of aromatic amino acid residues in AH₂. In some embodiments, the scaffold peptide has an aromatic amino acid residue at one or more of residue position numbers 1, 13 or 32 from the N-terminus of the peptide. In some embodiments, the one or more of residue position numbers 1, 13 or 32 from the N-terminus of the peptide is phenylalanine.

In some embodiments, oppositely charged amino acid residues in AH₁ and AH₂ interact with each other to form a plurality of salt bridges. A total of at least about eight intra-helix salt bridges may be formed between charged amino acids of each of AH₁ and AH₂. In some embodiments, one or more salt bridges are formed between residue numbers 3 and 4, residue numbers 3 and 6, residue numbers 4 and 7, residue numbers 7 and 10 and/or residue numbers 11 to 15 within AH₁ counting from the N-terminus of AH₁. In some embodiments, one or more salt bridges are formed between residue numbers 3 and 6, residue numbers 4 and 7, residue numbers 7 and 10, residue numbers 11 to 15 and/or residue numbers 15 and 18 within AH₂ counting from the N-terminus of AH₂.

In some embodiments, AH₁ and AH₂ each comprise at least two charged amino acid residues. The charged residues may be positioned at one or more of residue position numbers 4, 6, 10, 15, 23, 25, 29 or 34 from the N-terminus of the scaffold peptide. The charged residue may have a positive charge. The charged residue may be a lysine or an arginine.

In some embodiments, AH₁ and AH₂ each comprise at least two charged amino acid residues that form an intra-helical salt bridge, and one of the at least two charged amino acid residues is located at residue position numbers 3, 7, 11, 22, 26, 30, 37 from the N-terminus of the peptide. The charged residue may have a negative charge. The charged residue may be an aspartate (D) or a glutamate (E).

The AH₁ and AH₂ regions may each have an alpha helical structure. In some embodiments, the linker flexibly connects AH₁ and AH₂ so that AH₁ and AH₂ are folded against each other to form a bi-helical structure.

In some embodiments, AH₁ and AH₂ each comprise an amino acid sequence having a general formula from the N-terminus to the C-terminus, (Pho)ₐ-(Phi)_{b}-(Pho)_{c}-(Phi)_{d}-(Pho)ₑ-(Phi)_{f}-(Pho)_{g}-(Phi)ₕ-(Pho)ᵢ-(Phi)ⱼ, wherein a is 1-3, b is 2, c is 1, d is 2, e is 2, f is 2, g is 3, h is 1, i is 2 and j is 1, and Pho is any hydrophobic amino acid residue and Phi is any hydrophilic amino acid residue. In some embodiments, a is 2. In some embodiments, one of the hydrophobic amino acid residues in (Pho)_{g} is an aromatic amino acid residue.

In some embodiments, AH₁ and AH₂ each comprise an amino acid sequence having a general formula from the N-terminus to the C-terminus, (Pho)ₐ-Neg-Pos-(Pho)_{b}-Pos-Neg-(Pho)_{c}-Pos-Neg-(Pho)_{d}-Pos-(Pho)ₑ-Neg wherein a is 2, b is 1, c is 2, d is 3, e is 2, and Pho is any hydrophobic amino acid residue, Neg is any negatively charged amino acid residue, Pos is any positively charged amino acid residue, and one of the hydrophobic amino acid residues in (Pho)_{d} comprises an aromatic amino acid residue.

In some embodiments, AH₁ comprises an amino acid sequence represented by a general formula from the N-terminus to the C-terminus, Aro-Sma-Neg-Pos-Aro-Pos-Neg-Sma-Pho-Pos-Neg-Aro-Aro-Sma-Pos-Aro-Aro-Neg, and AH₂ comprises an amino acid sequence represented by a general formula from the N-terminus to the C-terminus, Sma-Sma-Neg-Pos-Pho-Pos-Neg-Sma-Pho-Pos-Neg-Aro-Aro-Sma-Pos-Pho-Aro-Neg, wherein Neg is any negatively charged amino acid residue, Pos is any positively charged amino acid residue, Aro is any aromatic amino acid residue and Sma is any small hydrophobic amino acid residue.

In some embodiments, one or more hydrophobic amino acid residues in the amphipathic scaffold peptide interacts with one or more hydrophobic amino acid residues in the at least one hydrophobic region of the protein in aqueous solution.

In some embodiments, the ratio of aromatic amino acid residues in the amino acid sequences of AH₁ and AH₂ is any one of 4:1, 3:1, 3:2 or 2:1. The aromatic amino acid residue may be phenylalanine (F) or tryptophan (W).

In some embodiments, the linker is at least one of a proline (P), glycine (G) or an alanine (A) amino acid residue.

In some embodiments, the amphipathic scaffold peptide has an amino acid sequence set forth in SEQ ID NO: 2. In some embodiments, the amphipathic scaffold peptide has an amino acid sequence of at least 80% sequence similarity to SEQ ID NO: 2. In some embodiments, the amphipathic scaffold peptide has the sequence of SEQ ID NO: 2, with at least one conservative amino acid substitution therein

In some embodiments, the amphipathic scaffold peptide or nanoscale particle has a solubility of at least 5 mg/mL in water.

In some embodiments, the number of inter-helix salt bridges formed between AH₁ and AH₂ is greater than about 40. In some embodiments, the number of inter-helix salt bridges formed between AH₁ and AH₂ is between about 45 and 70.

In some embodiments, the change in Gibbs free energy (ΔG) of the amphipathic scaffold peptide is at least 29 kcal/mol. In some embodiments, the change in Gibbs free energy (ΔG) of AH₁ is less than about 20 kcal/mol. In some embodiments, the change in Gibbs free energy (ΔG) of AH₁ is at least 12 kcal/mol. In some embodiments, the change in Gibbs free energy (ΔG) of AH₁ is between about 12 kcal/mol and about 19 kcal/mol. In some embodiments, the change in Gibbs free energy (ΔG) of AH₂ is greater than 17.5 kcal/mol. In some embodiments, the change in Gibbs free energy (ΔG) of AH₂ is at least 16 kcal/mol. In some embodiments, the change in Gibbs free energy (ΔG) of AH₂ is between 17.5 kcal/mol and about 21 kcal/mol.

In some embodiments, the hydrophobic moment of the amphipathic scaffold peptide is at least about 20.05 kcal/mol. In some embodiments, the hydrophobic moment of AH₁ is at least 19.60 kcal/mol. In some embodiments, the hydrophobic moment of AH₁ is between 20 kcal/mol to about 22 kcal/mol. In some embodiments, the hydrophobic moment of AH₂ is less than 21 kcal/mol. In some embodiments, the hydrophobic moment of AH₂ is between about 16 kcal/mol to 21 kcal/mol.

In some embodiments, the absolute hydrophobic moment of the amphipathic scaffold peptide is less than about 7.000 A*kT/e. In some embodiments, the absolute hydrophobic moment of the amphipathic scaffold peptide is less than 6.000 A*kT/e. In some embodiments, the absolute hydrophobic moment of the amphipathic scaffold peptide is about 5.792 A*kT/e.

In some embodiments, the absolute surface electropotential of the amphipathic scaffold peptide is between about 4.500 kT/e and about 5.000 kT/e. In some embodiments, the absolute surface electropotential of the amphipathic scaffold peptide is greater than about 5.100 kT/e. In some embodiments, the absolute surface electropotential of the amphipathic scaffold peptide is about 5.234 kT/e.

In some embodiments, a solvent accessible area of the amphipathic scaffold peptide is greater than about 3400 A².

In some embodiments, the total number of amino acid residues in the amphipathic scaffold peptide is in a range of about 30 to 45.

In some embodiments, a cysteine residue is coupled to the N-terminus of the amphipathic scaffold peptide. In some embodiments, an affinity tag attached to one or both of the N-terminus and the C-terminus of the scaffold peptide. The affinity tag may be a histidine-tag or a biotinylation-tag. The histidine-tag may optionally be a hexahistidine tag. In some embodiments, a fluorescent or luminescent label is coupled to at least one of the N-terminus and/or the C-terminus. The fluorescent label may be any one of: dansyl, maleimide, fluorescein isothiocyanate (FITC), ortho-aminobenzoic acid (Abz), dinitrophenol (DNP), 4-(dimethylaminoazo)benzene-4-carboxylic acid (DABCYL), 5-((2-Aminoethyl)amino)naphthalene-1-sulfonic acid (EDANS), cyanine dyes, ATTO, Alexa or fluorescein amidite (FAM). In an example embodiment, a fluorescein amidite (FAM).fluorescent label is coupled to the N-terminus of the scaffold peptide. In another example embodiment, a 1-pyrenebutyric acid label is coupled to the C-terminus of the scaffold peptide.

In some embodiments, an oligonucleotide label is coupled to at least one of the N-terminus and/or the C-terminus, or to a cysteine residue of the scaffold peptide.

Aspects of the invention relate to a nanoscale particle. The nanoscale particle has an amphipathic scaffold peptide according to embodiments of this invention and a protein having at least one hydrophobic region. The amphipathic scaffold peptide may be self-assembled at the at least one hydrophobic region of the protein in aqueous solution. In some embodiments, the amphipathic scaffold peptides are assembled in a tilted orientation at the at least one hydrophobic region of the protein. The protein may be any one of an alpha-helical membrane protein, a beta-barrel membrane protein, a transmembrane protein (TMS), a monomeric membrane protein or an oligomeric membrane protein.

Aspects of the invention relate to a method of stabilizing a membrane protein. The method may include obtaining the membrane protein and combining the membrane protein with an amphipathic scaffold peptide according to an embodiment of the invention. A plurality of the amphipathic scaffold peptide may be added. The step of combining the membrane protein with the amphipathic scaffold peptide may result in the amphipathic scaffold peptide self-assembling around one or more hydrophobic regions of the membrane protein to yield a nanoscale particle. In some embodiments, an exogenous lipid may be added in the combining step.

The step of obtaining the membrane protein may involve isolating the membrane protein from a membrane by adding a solubilizing agent to a solution containing the membrane and the membrane protein. The step of adding a solubilizing agent to the solution may involve adding a detergent to the solution.

The step of combining the membrane protein with the amphipathic scaffold peptide may be performed by using any one of size-exclusion chromatography, gel electrophoresis, affinity chromatography, density gradient centrifugation, or by removing unbound/non-incorporated lipids and/or peptides and/or detergent by: dialysis, detergent-binding biobead separation, magnetic bead separation, concentrators, or membrane filtration.

The method optionally involves a first step of determining an optimal reconstitution ratio of the amphipathic scaffold peptides to membrane protein before the combining step. The combining step may then optionally involve adding the determined optimal reconstitution ratio of amphipathic scaffold peptides to the membrane protein. The step of determining the optimal amphipathic scaffold peptide to membrane protein reconstitution ratio may be performed using gel electrophoresis.

Aspects of the invention relate to a method of use of an amphipathic scaffold peptide and a nanoscale particle according to an embodiment of the invention. Examples of use of the amphipathic scaffold peptide and/or the nanoscale particle include the purification of membrane proteins, the study of membrane proteins, the production of antibodies, the creation of membrane protein libraries, drug deliver, the testing and discovery of membrane proteins and binding interactors, and the formulation of vaccines and cosmetics.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic diagram showing how amphipathic scaffold peptides can be used to stabilize a membrane protein without the need for exogenous detergent.

FIG. 2A is a side view of a ribbon model of a nanoscale particle being a membrane protein stabilized by a plurality of amphipathic scaffold peptides according to an example embodiment. FIG. 2B is a top view of FIG. 2A. FIG. 2C is a side view of the ribbon model of FIG. 2A with the membrane protein omitted for clarity. FIG. 2D is a top view of FIG. 2C. FIG. 2E is a perspective view of a cryo-electron microscopy (cryo-EM) image of a membrane protein stabilized by a plurality of amphipathic scaffold peptides according to an example embodiment. FIG. 2F is a ribbon model of the cryo-EM image of FIG. 2E. FIG. 2G is a perspective view of the cryo-EM image of FIG. 2E with the membrane protein replaced by a ribbon model. FIG. 2H is a top view of FIG. 2G.

FIG. 3 is a schematic diagram showing the putative structure of an example embodiment of an amphipathic scaffold peptide, with all of the amino acid side chains illustrated schematically. The lower panel image is rotated by 180° about a vertical axis relative to the upper panel.

FIG. 4 is a schematic diagram of the embodiment of FIG. 3, showing only the hydrophobic amino acid side chains. The lower panel image is rotated by 90° about a horizontal axis relative to the upper panel.

FIG. 5 is a schematic diagram of the embodiment of FIG. 3, showing only the hydrophilic amino acid side chains. The lower panel image is rotated by 90° about a horizontal axis relative to the upper panel.

FIG. 6A is a schematic diagram of the embodiment of FIG. 3, showing only the charged amino acid side chains. White indicates a neutral charge, grey indicates a negative charge, and black indicates a positive charge. FIG. 6B is a model of an example embodiment of an amphipathic scaffold peptide, showing the location of the alternating positively and negatively charged regions of the surface of the peptide with shading.

FIG. 7A is a schematic diagram of an example embodiment of an amphipathic scaffold peptide showing the location of putative intra-helical salt bridges believed to form between charged amino acid residues in the peptide in dashed lines together with the corresponding sequence (corresponding to SEQ ID NO: 2) of the amphipathic scaffold peptide and the locations of the amino acid residues that interact to form the salt bridges. FIG. 7B illustrates the amino acid sequence of SEQ ID NO: 2 and the locations of the amino acid residues that interact to form the salt bridges as shown in FIG. 7A with the amphipathic scaffold peptide omitted for clarity.

FIG. 8A is a helical wheel diagram of a first helix region of a scaffold peptide. FIG. 8B is a helical wheel diagram of a second helix region of the scaffold peptide.

FIG. 9A is a table showing example amino acid residues that can be included at each of positions 1-19 of the amino acid sequence of the amphipathic scaffold peptide in some embodiments. FIG. 9B is a table showing example amino acid residues that can be included at each of positions 20-37 of the amino acid sequence of the amphipathic scaffold peptide in some embodiments.

FIG. 10A is a flowchart of a method for reconstituting a membrane protein using a scaffold peptide according to an example embodiment. FIG. 10B is a schematic diagram illustrating the hypothetical efficacy of membrane protein reconstitution. The thickness of the lines corresponds with the extent of favourability of a reaction.

FIG. 11 is a schematic diagram illustrating a method of membrane protein isolation according to an example embodiment, showing how the amphipathic scaffold peptides self-assemble to stabilize and solubilize the membrane protein.

FIG. 12 is a schematic diagram illustrating a method of determining an optimal reconstitution ratio for scaffold peptide:membrane protein to form a nanoscale particle or peptidisc according to an example embodiment.

FIG. 13 is a schematic diagram illustrating a reconstitution method of forming peptidiscs using size-exclusion chromatography according to an example embodiment.

FIG. 14 is a schematic diagram illustrating a reconstitution method of forming peptidiscs using a density gradient centrifugation method according to an example embodiment.

FIG. 15 is a schematic diagram illustrating a reconstitution method of forming peptidiscs using affinity chromatography according to an example embodiment.

FIG. 16 is a schematic diagram illustrating a method of forming peptidiscs using affinity chromatography according to an example embodiment.

FIG. 17A are peptide models computed by a 3D-hydrophobic moment peptide calculator showing the direction of hydrophobic moment of NSPᵣ and NSP (line near the centre of each helical model). FIG. 17B shows measurements of turbidity of NSPᵣ and NSP scaffold peptide suspensions as compared to a distilled water (dH₂O) control. FIG. 17C shows calculations of electropotential and hydrophobic moment of NSPᵣ and NSP.

FIG. 18A illustrates a typical size-exclusion chromatography of MalFGK₂ in stabilized by amphipathic scaffold peptides to form a peptidisc complex in one example embodiment (MalFGK₂-NSPᵣ). FIG. 18B are results from CN-PAGE and BN-PAGE analysis of MalFGK₂ in detergent micelle (DDM), nanodisc (MSP1D1), and peptidisc (NSPᵣ,). FIG. 18C shows maltose-dependent ATPase activity of MalFGK₂ (0.5 μM) reconstituted in detergent (DDM), proteoliposomes (PL), peptidiscs (NSPᵣ,), and nanodiscs (MSP1D1) obtained at 30° C. in the presence or absence of MalE (2.5 μM).

FIG. 19A illustrates the results of a CN-PAGE analysis from loading a mixture of NSPᵣ and MalFGK₂ in a buffer containing a low amount of detergent (about 0.008% DDM). FIG. 19B illustrates the results of a CN-PAGE analysis from loading a mixture of NSPᵣ and FhuA in a buffer containing a low amount of detergent. FIG. 19C illustrates the results of a CN-PAGE analysis from loading a mixture of NSPᵣ and OmpF₃ in a buffer containing a low amount of detergent. FIG. 19D illustrates the results of a CN-PAGE analysis from loading a mixture of NSPᵣ and SecEYG in a buffer containing a low amount of detergent. FIG. 19E is a graph illustrating the reconstitution efficiency of FhuA and the half-maximal reconstitution ratio (RR50) for the calculation of the half-maximal reconstitution ratio (RR50) of FhuA. FIG. 19F illustrates the RR50 values for the target proteins SecEYG, FhuA, MalFGK₂ and OmpF₂.

FIG. 20A is a schematic diagram illustrating the “on-beads” reconstitution method of forming peptidiscs. FIG. 20B shows SDS-PAGE analysis of a His-tagged MalFGK₂ complex purified following conventional detergent method and “on-beads” reconstitution method. FIG. 20C shows a Native-PAGE analysis (BN and CN) of a His-tagged MalFGK₂ complex purified following conventional detergent method and “on-beads” reconstitution method.

FIG. 21A shows the absorbance of the BRC (1 μM) at 803 nm in detergent solution (0.03% LDAO) and in peptidisc. FIG. 21B shows the absorbance of the BRC at 803 nm after incubation at 65° C. over time. FIG. 21C is a graph showing the calculated half-life of the BRC in peptidisc and LDAO at 65° C.

FIG. 22A illustrates the results of a 15% SDS-PAGE analysis of MalFGK₂ in peptidisc or DDM. FIG. 22B illustrates a standard curve derived from NSPᵣ titration measurement, and average intensity of NSPᵣ fluorescence from MalFGK₂ peptidisc. FIG. 22C is a western blot showing FhuA-peptidisc reconstituted into NSP_{rbio}. The biotin label was attached to the N-terminus of NSPᵣ. FIG. 22D illustrates a standard curve derived from NSPᵣ titration measurement, and average intensity of NSPᵣ fluorescence from FhuA-peptidisc. FIG. 22E illustrates the results of a 15% SDS-PAGE analysis of BRC in peptidisc. FIG. 22F illustrates a standard curve derived from NSPᵣ titration measurement, and average intensity of NSPᵣ fluorescence from BRC in peptidisc.

FIG. 23A shows the results of a Malachite green assay showing the phospholipid content of each of FhuA-peptidisc, BRC-peptidisc and MalFGK₂-peptidisc. FIG. 23B shows the results of a thin layer chromatography analysis of lipid extracts obtained from 10 μg MalFGK₂ peptidisc, 10 μg FhuA peptidiscs and 20 μg BRC peptidiscs, as well as pure lipid standards Cardiolipin (CL), 1,2-dioleoyl-sn-glycero-3-phosphoglycerol (PG), and 1,2-dioleoyl-sn-glycero-3-phosphoethanolamine (PE).

FIG. 24A illustrates the results of a multi-angle light scattering analysis of MalFGK₂ reconstituted in peptidisc. FIG. 24B illustrates the results of a BN-PAGE showing the structural stability of MalFGK₂-NSPᵣ over time upon incubating the MalFGK₂ peptidisc overtime.

FIG. 25A illustrates a typical SEC profile of FhuA reconstituted in peptidisc (FhuA-NSPᵣ) using an ‘on-column’ reconstitution protocol. FIG. 25B illustrates the results of a CN-PAGE of the FhuA transporter reconstituted in nanodiscs (FhuA-MSP_{L156}) or peptidiscs (FhuA-NSPᵣ), which was incubated with the C-terminal TonB₂₃₋₃₂₉ fragment (2 μg) or with colicin M (5 μg), with or without ferricrocin.

FIG. 26A illustrates the results of a CN-PAGE showing the SecYEG complex (2.5 μg) incubated with each of NSPᵣ, MSP1D1, MSP1D1E3 (1.25 μg each) in Buffer A+0.02% DDM. FIG. 26B illustrates the results of a CN-PAGE showing each of NSPr, MSP1D1, MSP1D1E3 (1.25 μg each) in Buffer A+0.02% DDM. FIG. 26C is a schematic diagram representing possible reconstitution products of the SecYEGₙ complex into MSP1D1 (labelled as Nanodisc) and NSFᵣ (labelled as Peptidisc).

FIG. 27A illustrates the results of absorbance scans of the BRC complex (1 μM) in peptidisc after incubation at 65° C. for up to 1 hour and incubation at 90° C. FIG. 27B illustrates the results of absorbance scans of the BRC (1 μM) after incubation at 65° C. in 0.03% LDAO for up to 4 minutes. FIG. 27C shows the fluorescence measurements (700 nm; excitation at 680 nm) of the BRC (1 μM) after incubation in peptidisc, 0.1% LDAO, 0.02% DDM, and 0.1% SDS for 5 minutes at varying temperatures. FIG. 27D shows the fluorescence measurements (700 nm; excitation at 680 nm) of the BRC (1 μM) reconstituted into MSP1D1 (1:2 BRC:MSP₁D₁ molar ratio), SMA (0.1%), Proteoliposomes (1:1600:400 BRC:DOPC:DOPG), and peptidiscs). FIG. 27E illustrates the calculated melting temperatures (Tₘ) of the BRC complex with each of NSPᵣ, SMA, MSP₁D₁, PL, DDM, LDAO, and SDS. FIG. 27F illustrates of the results of the BN-PAGE analysis (left panel) and SDS-PAGE analysis (right panel) of the reconstituted BRC fractions.

FIG. 28A illustrates the results of a SDS-PAGE analysis of detergent solubilized E. coli crude membrane before and after reconstitution into peptidiscs. FIG. 28B and FIG. 28C show the SEC-fractionation of DDM extract and peptidisc-stabilized protein library prepared from DDM extract. FIG. 28D shows a comparison between the number of proteins identified in fraction #12 of the DDM extract and the peptidisc-stabilized protein library prepared from the DDM extract. FIG. 28E illustrates the results of CN-PAGE analysis of crude membrane solubilized in DDM (Lane 1) or in peptidiscs (Lane 2). FIG. 28F illustrates the peptidisc library containing overexpressed MsbA (Lane 1), then bound to Ni-NTA beads, washed in Buffer A (Lane 2), and eluted in Buffer A+250 mM imidazole (Lane 3). FIG. 28G illustrates the results of a CN-PAGE analysis of MsbA purified from the DDM extract (Lane 1) or purified from the peptidisc-stabilized protein library (Lane 2).

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein, the term “hydrophobic biomolecules” includes hydrophobic proteins, including membrane proteins, lipids, lipid-based proteins, and lipid-protein complexes. The term “hydrophobic proteins” includes proteins having at least one significantly hydrophobic face, and includes membrane proteins.

As used herein, the term “membrane proteins” includes transmembrane proteins, integral monotopic membrane proteins, peripheral membrane proteins, amphitropic proteins in a lipid-bound state, lipid-anchored proteins, chimeric proteins with a fused hydrophobic and/or transmembrane domain, and the like.

In some embodiments, hydrophobic proteins, including membrane proteins, are from eukaryotes. In some embodiments, the hydrophobic proteins, including membrane proteins, are from prokaryotes.

As used herein, the term “amino acid” means any naturally occurring amino acid or non-naturally occurring or synthetic amino acid, including but not limited to, amino acids in either the L-form or the D-form. In some embodiments, all of the amino acids in the sequences listed below are L-amino acids. Examples of amino acids that may be used in various embodiments include the twenty standard amino acids, as well as other amino acids such as citrulline, hydroxyproline, norleucine, 3-nitrotyrosine, nitroarginine, ornithine, naphtylalanine, methionine sulfoxide, methionine sulfone, or the like.

As used herein, the abbreviation “Pho” means any hydrophobic amino acid residue. A hydrophobic amino acid includes Alanine (A), Valine (V), Isoleucine (I), Leucine (L), Proline (P), Phenylalanine (F), Methionine (M) Tryptophan (W), and any variants and chemically modified derivatives thereof or artificial amino acids that are hydrophobic.

As used herein, the abbreviation “Phi” means any hydrophilic amino acid residue. A hydrophilic amino acid residue includes Arginine (R), Asparagine (N), Aspartate (D), Cysteine (C), Glutamate (E), Glutamine (Q), Glycine (G), Histidine (H), Lysine (K), Serine (S), Threonine (T), Tyrosine (Y), Selenocysteine (U) and any variants and chemically modified derivatives thereof or artificial amino acids that are hydrophilic.

As used herein, the abbreviation “Pos” means any positively charged amino acid residue. A positively charged amino acid includes K, R, or H and any variants and chemically modified derivatives thereof or artificial amino acids that are positively charged.

As used herein, the abbreviation “Neg” means any negatively charged amino acid residue. A negatively charged amino acid residues includes D or E and any variants and chemically modified derivatives thereof or artificial amino acids that are negatively charged.

As used herein, the abbreviation “Aro” means any amino acid residue that includes an aromatic ring. An aromatic amino acid residue includes any one of F, Y, W, and any variants and chemically modified derivatives thereof or artificial amino acids that have a side chain containing an aromatic moiety.

As used herein, the abbreviation “Sma” means any small hydrophobic amino acid residues including A, P, G, C or any small polar amino acid residues including S, T, D, N and any variants and chemically modified derivatives thereof.

As used herein, the term “variant” refers to a peptide having an amino acid sequence that differs from a reference peptide by one or more insertions, deletions, or substitutions of one or more amino acid residues relative to a reference molecule. As used herein the term “variant having a conservative amino acid substitution” means a peptide having an amino acid sequence that differs from a reference peptide by one or more conservative substitutions of one or more amino acid residues relative to a reference molecule. Amino acid substitutions that are considered to be “conservative” include any of:
Interchanging any one of A, V, L, or I, or an artificial amino acid substantially similar thereto;
Interchanging any one of S, C, U, T, or M, or an artificial amino acid substantially similar thereto;
Interchanging any one of F, Y, or W, or an artificial amino acid substantially similar thereto;
Interchanging any one of D or E or an artificial amino acid substantially similar thereto;
Interchanging any one of N or Q or an artificial amino acid substantially similar thereto;
Interchanging any one of H, K or R or an artificial amino acid substantially similar thereto.

Some embodiments of the invention relate to the field of a scaffold peptide for use in the isolation, purification and/or characterization of hydrophobic biomolecules such as membrane proteins. The scaffold peptide has amphipathic properties having both hydrophilic and hydrophobic regions in its structure. In some embodiments, the scaffold peptide is asymmetrical, i.e. the scaffold peptide is formed from a first alpha helical portion connected to a second alpha helical portion via a linker, and one of the first or second alpha helical portions contains a higher number of aromatic amino acid residues than the second or first alpha helical portion, respectively. Without being bound by theory, it is believed that the asymmetrical peptide facilitates intra-peptide associations with surrounding water molecules. The asymmetrical scaffold peptide is thus more water-soluble than would be for an equivalent symmetrical scaffold peptide (i.e. a scaffold peptide having equal numbers of aromatic amino acid residues in both of the first and second alpha helical portions).

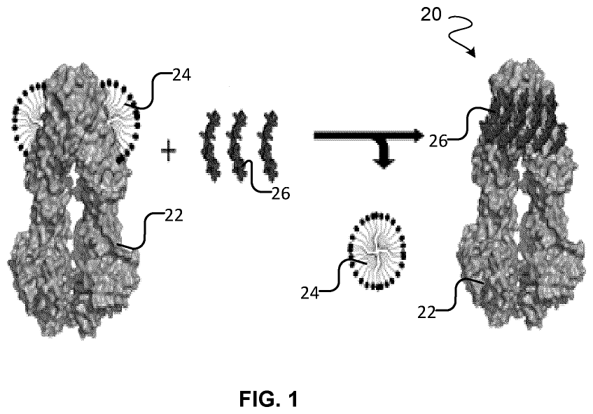

With reference to FIG. 1, an example embodiment illustrating how a plurality of amphipathic scaffold peptides 26 can be used to stabilize a membrane protein 22 in aqueous solution by interacting with the transmembrane domains of the membrane protein is shown. Initially, as shown on the left hand side of FIG. 1, membrane protein 22 is stabilized and isolated in a traditional manner using a solubilizing agent such as a detergent 24. In some embodiments of the present invention, a plurality of amphipathic scaffold peptides 26 are introduced into the solution containing the membrane protein, and the detergent 24 is removed in any suitable manner. The plurality of the amphipathic scaffold peptides 26 self-assembles onto the membrane protein. The result is a membrane protein 22 complexed with a plurality of the amphipathic scaffold peptides 26 to form a stabilized membrane protein complex referred to herein as a “peptidisc” 20.

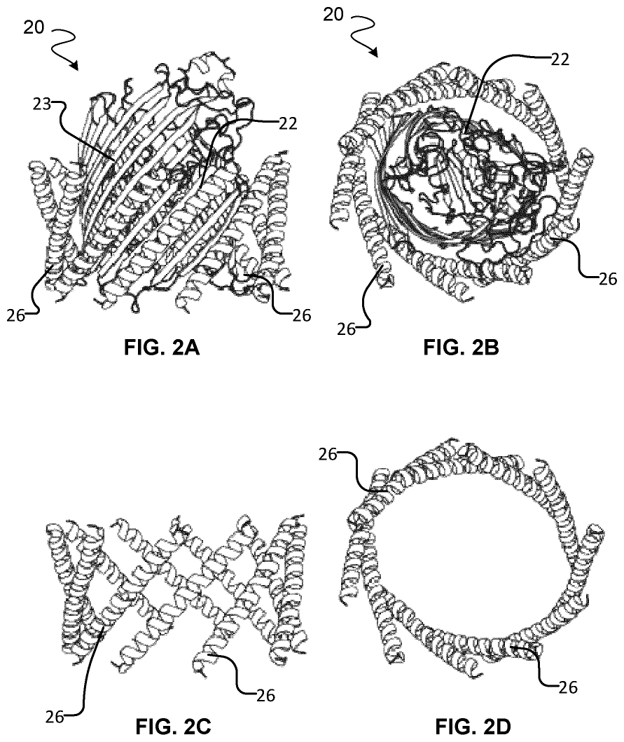

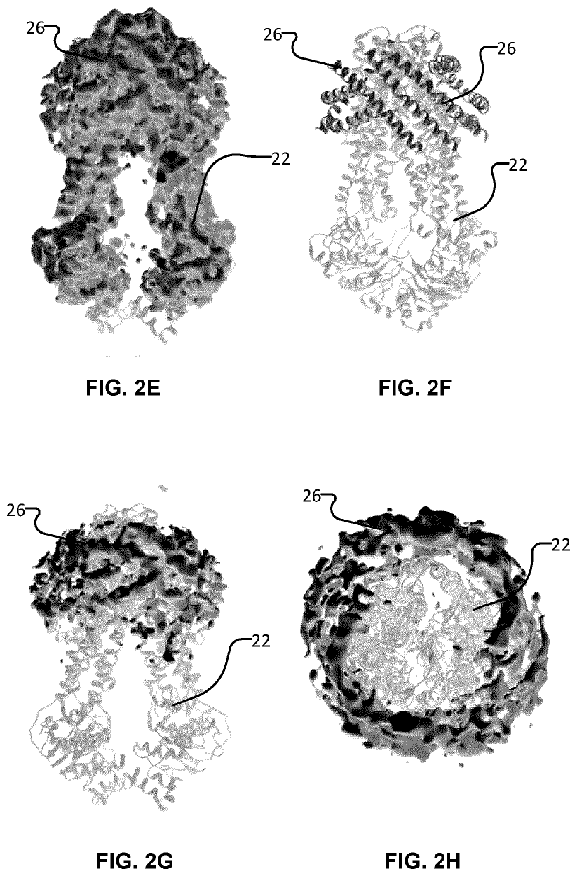

With reference to FIGS. 2A and 2B, when assembled to form a peptidisc 20, the amphipathic scaffold peptide acts to shield the hydrophobic regions of the membrane protein 23 from the aqueous solution, thereby solubilizing and stabilizing the membrane protein. Without being bound by theory, it is believed that the plurality of amphipathic scaffold peptides self-assemble and contact the hydrophobic regions 23 of the membrane protein or the alkyl chains of annular lipids (i.e., the lipids which preferentially bind to the surface of membrane proteins). After binding, the amphipathic scaffold proteins begin to shift on the surface of the membrane protein at an angle with respect to the vertical axis of the membrane protein. The amphipathic scaffold peptides may continue to shift on the surface of the membrane protein until they have each reached an optimal binding site on the membrane protein.

Without being bound to any theory, the inventors believe that the amphipathic scaffold peptides bind the membrane protein in a tilted orientation on transmembrane portions of the membrane protein. A tilted orientation is believed to facilitate optimal binding and stoichiometry of the amphipathic scaffold peptide to the target membrane protein. The tilted orientation of the amphipathic scaffold peptides on the target membrane protein is best illustrated in FIGS. 2C and 2D. The self-assembly and specifically the tilted orientation of the amphipathic scaffold peptides on the target membrane protein is believed to allow the amphipathic scaffold peptides to fit membrane proteins of any type, size and structure. Consequently, amphipathic scaffold peptides according to various embodiments can be used to stabilize any type of membrane protein.

Without being bound by theory, the basis for believing that the amphipathic scaffold peptides adopt a tilted orientation on the transmembrane portions of the membrane protein is as follows. Based on prior observations, in ApoA1 lipid nanodiscs, the two scaffold proteins arrange themselves in an anti-parallel “double belt” configuration (S. Bibow S, Y. Polyhach, C. Eichmann, C N, Chi, J. Kowal, S. Albiez, R A, McLeod. H. Stahlberg, G. Jeschke, P. Güntert P, R. Riek, Nature Structural & Molecular Biology, 2017, 24:187-193.). If NSPᵣ were also arranged in a double belt, then the peptide to protein ratio would expectedly vary by factor of 2, as an odd number of scaffold peptides would leave part of the protein exposed to the environment. However, the native mass spectrometry profiles for FhuA and BRC stabilized by NSPᵣ observed by the inventors indicate peptidisc populations which can differ in mass by 1 peptide only, suggesting an arrangement that is flexible. It is possible that the amphipathic scaffold peptides could be arranged in an orthogonal “picket fence” orientation as proposed for lipopeptides, nanostructured [beta]-sheet peptides, and single helix ApoA1 mimetic peptides (H. Tao, S. C. Lee, A. Moeller, R. T. Roy, F. Y. Siu, J. Zimmerman, R. C. Stevens, C. S. Potter, B. Carragher and Q. Zhang. Nat. Methods. 2013, 10, 759-761.; G. G. Privé, Curr. Opin. Struct. Biol., 2009, 379-385.; R. M. Islam, M. Pourmousa, D. Sviridov, S. M. Gordon, E. B. Neufeld, L. A. Freeman, B. S. Perrin, R. W. Pastor, A. T. Remaley, Scientific Reports, 2018. 8:2956.). However, the length of the NSPᵣ amphipathic scaffold peptide used to obtain these observations (37 amino acids) is too long to be orthogonal while maintaining contact with hydrophobic parts of the protein or alkyl chains of annular lipids. Hence, it is believed the amphipathic scaffold peptides lie in a tilted orientation, i.e. vertically aligned on the transmembrane portion of the membrane protein but with the vertical axis of the amphipathic scaffold peptides shifted slightly relative to the vertical axis of the membrane protein, assuming that the membrane defines a horizontal axis. A tilted orientation would facilitate optimal binding and stoichiometry as the peptide shifts in angle of association, adapting to best fit the target membrane protein template. It is believed that this tilted orientation explains how the amphipathic scaffold peptides can be used to reconstitute so many different membrane proteins, as the scaffold can shift its angle of association to best fit the membrane protein template.

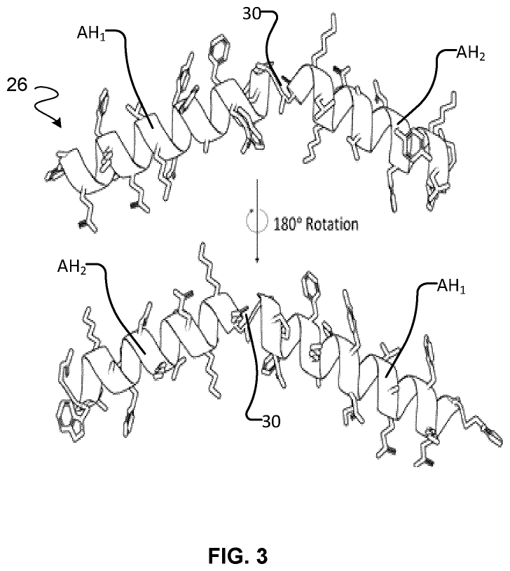

In some embodiments, as shown in FIGS. 3 to 7, the amphipathic scaffold peptide has two alpha-helical regions AH₁ and AH₂ joined by a flexible linker, illustrated as 30. In some embodiments, the amphipathic scaffold peptide has an amino acid sequence having a general formula, from the N-terminus to the C-terminus: AH₁-linker-AH₂ wherein AH₁ and AH₂ are peptide sequences that are independently between 15 and 30 amino acids in length, including any value therebetween e.g., 16, 17, 18, 19, 20, 21, 22, 23, 24, 25, 26, 27, 28 or 29 amino acids in length. In an example embodiment, AH₁ and AH₂ each comprise 18 amino acids. In some embodiments, either or both of AH₁ and AH₂ has a core alpha-helical segment of approximately 18 amino acids in length and one or more additional amino acids present at the N-terminus of AH₁ or at the C-terminus of AH₂. For example, in one embodiment a hexahistidine tag (i.e. H-H-H-H-H-H) is provided at the N-terminus of AH₁, to facilitate the purification of the amphipathic scaffold peptides and/or membrane proteins stabilized using the amphipathic scaffold peptides.

In some embodiments, the linker 30 is a flexible linker. In some embodiments, the linker 30 comprises one or more amino acid residues to form a flexible linker that allows the amphipathic scaffold peptide to fold back on itself so that AH₁ and AH₂ can interact as described below. Any suitable amino acid residue or series of amino acid residues may be used to form the linker 30. In some embodiments, the linker has 1, 2, 3, 4, 5, 6, 7, 8, 9, 10 or more amino acid residues. In some embodiments, the linker is a proline residue. In some embodiments, the linker is a glycine residue. In some embodiments, the linker is an alanine residue. In some embodiments, the linker is any small hydrophobic amino acid residue. In some embodiments, the linker is any amino acid residue or combination of amino acid residues that allows the peptide to bend so that AH₁ and AH₂ can interact with one another via their complimentary amphipathic structures as described below.

In some embodiments, linker 30 can be provided in any other suitable manner. For example, in some embodiments, a cysteine residue could be provided at either the N-terminus or the C-terminus of AH₁, and a corresponding cysteine residue could be provided at the C-terminus or N-terminus of AH₂ and the linker 30 formed by the formation of a disulfide bond between the two cysteine residues.

In some embodiments, the number of amino acid residues in the scaffold peptide, i.e., AH₁ plus AH₂ is greater than about eighteen. The length of the amphipathic scaffold peptide may impact its binding affinity onto the target membrane protein. A shorter amphipathic scaffold peptide (e.g. having about eighteen amino acid residues in the sequence or about nine amino acid residue in each of AH₁ and AH₂) may lack the degree of hydrophobicity required to remain stably bound to the target membrane protein, which could require the continuous presence of an excess of amphipathic scaffold peptide in order to prevent aggregation of the membrane protein or allow for removal of solubilizing agents such as detergent. The inventors believe that an ideal amphipathic scaffold peptide will remain stably bound to the target membrane protein upon prolonged incubation even at elevated temperatures. The length of the scaffold peptide may also impact the dissociation of the solubilizing agent (i.e., detergent) from the target membrane protein to allow the scaffold peptide to bind to the target membrane protein. In some embodiments, the total number of amino acid residues in the amphipathic scaffold peptide is in a range of about 30 to about 45, including any value therebetween e.g. 31, 32, 33, 34, 35, 36, 37, 38, 39, 40, 41, 42, 43, or 44. In some embodiments, the total number of amino acid residues in the scaffold peptide is in a range of about 35 to about 38. In some embodiments, the total number of amino acid residues in the scaffold peptide is 37.

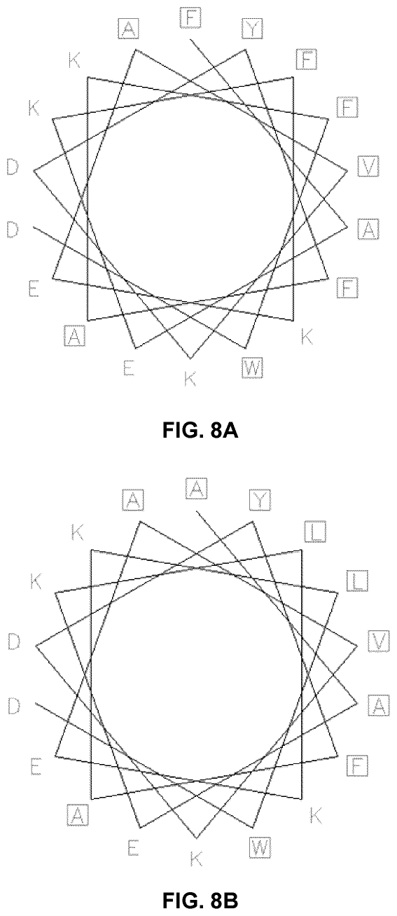

Without being bound by theory, it is believed the amphipathic scaffold peptide forms a bi-helical structure in solution. AH₁ and AH₂ may each be any amino acid sequence that forms a helical secondary structure. In some embodiments, the helical secondary structure of AH₁ and AH₂ includes opposing hydrophobic and hydrophilic faces. For example, as can be seen in FIGS. 8A and 8B which are helical wheel models of AH₁ and AH₂, respectively, in which amino acids having hydrophobic side chains are highlighted in square boxes while polar or charged amino acids are not, AH₁ shown in FIG. 8A has a hydrophilic surface towards the left side of the image as drawn and a hydrophobic surface towards the right side of the image as drawn. AH₂ shown in FIG. 8B similarly has a hydrophilic surface towards the left side of the image as drawn and a hydrophobic surface towards the right side of the image as drawn.

In some embodiments, AH₁ and AH₂ each independently have an amino acid sequence having alternating hydrophilic and hydrophobic regions. The hydrophobic regions of the amphipathic scaffold peptide facilitate the binding of the scaffold peptide to the hydrophobic transmembrane surface of the target membrane protein. The hydrophilic regions of the scaffold protein allow the non-soluble target membrane protein to become water-soluble upon binding. This is because the stabilized membrane protein complex can form hydrogen bonds with the surrounding water molecules via the hydrophilic amino acid side chains on the amphipathic scaffold peptides. In some embodiments, AH₁ and AH₂ comprises an amino acid sequence having from N-terminal to C-terminal a general formula: (Pho)ₐ-(Phi)_{b}-(Pho)_{c}-(Phi)_{d}-(Pho)ₑ-(Phi)_{f}-(Pho)_{g}-(Phi)ₕ-(Pho)ᵢ-(Phi)ⱼ, wherein a is 1-3, b is 2, c is 1, d is 2, e is 2, f is 2, g is 3, h is 1, i is 2 and j is 1. In some embodiments, Phi is either a positively charged amino acid residue or a negatively charged amino acid residue. In some embodiments, one or more of the hydrophobic residues in (Pho)_{d} includes an aromatic amino acid residue. In some embodiments, one or more of the hydrophobic residues in (Pho)_{g} includes an aromatic amino acid residue.

In some embodiments, AH₁ and AH₂ each independently have an amino acid sequence having a general formula: (Pho)ₐ-Neg-Pos-(Pho)_{b}-Pos-Neg-(Pho)_{c}-Pos-Neg-(Pho)_{d}-Pos-(Pho)ₑ-Neg wherein a is 2, b is 1, c is 2, d is 3, e is 2.

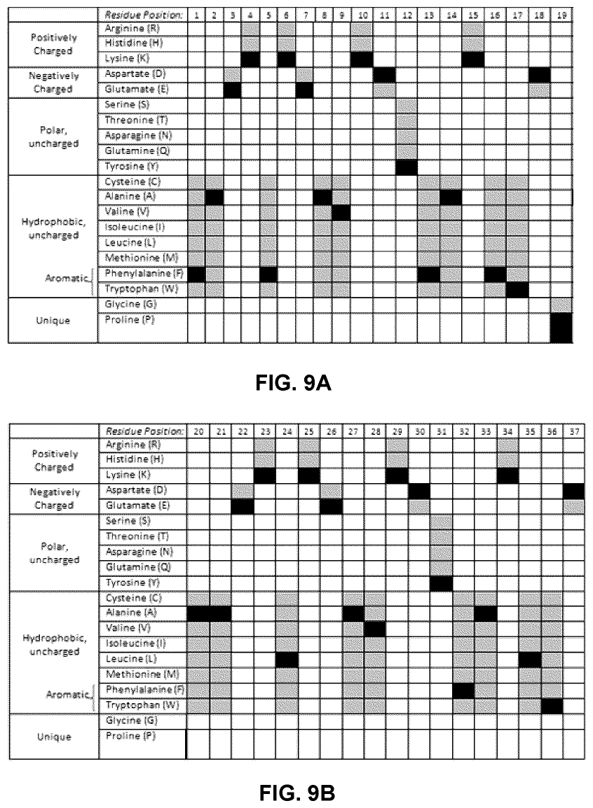

In some embodiments, AH₁ has the following sequence listed in Table 1 or shaded in FIG. 9A from N-terminal to C-terminal, with each amino acid residue at each of the indicated positions 1-18 being selected from one of the amino acid residues listed in the corresponding column.

TABLE 1
Possible Amino Acid Residues in Each Position Within AH₁.

| # | 1 | 2 | 3 | 4 | 5 | 6 | 7 | 8 | 9 | 10 | 11 | 12 | 13 | 14 | 15 | 16 | 17 | 18 |
|---|---|---|---|---|---|---|---|---|---|---|---|---|---|---|---|---|---|---|
| N-ter | F | A | E | K | F | K | E | A | V | K | D | Y | F | A | K | F | W | D |
| | C | C | D | R | C | R | D | C | C | H | E | S | C | C | H | C | C | E |
| | A | V | | H | A | H | | V | A | R | | T | A | V | R | A | A | |
| | V | I | | | V | | | I | I | | | N | V | I | | V | V | |
| | I | L | | | I | | | L | L | | | Q | I | L | | I | I | |
| | L | M | | | L | | | M | M | | | | L | M | | L | L | |
| | M | F | | | M | | | F | F | | | | M | F | | M | M | |
| | W | W | | | W | | | W | W | | | | W | W | | W | F | |

In some embodiments, AH₂ has the following sequence listed in Table 2 or shaded in FIG. 9B from N-terminal to C-terminal, with each amino acid residue at each of the indicated positions 1-18 being selected from one of the amino acid residues listed in the corresponding column.

TABLE 2
Possible Amino Acid Residues in Each Position Within AH₂.

| # | 1 | 2 | 3 | 4 | 5 | 6 | 7 | 8 | 9 | 10 | 11 | 12 | 13 | 14 | 15 | 16 | 17 | 18 |
|---|---|---|---|---|---|---|---|---|---|---|---|---|---|---|---|---|---|---|
| N-ter | A | A | E | K | L | K | E | A | V | K | D | Y | F | A | K | L | W | D |
| | C | C | D | H | C | H | D | C | C | H | E | S | C | C | H | C | C | E |
| | V | V | | R | A | R | | V | A | R | | T | A | V | R | A | A | |
| | I | I | | | V | | | I | I | | | N | V | I | | V | V | |
| | L | L | | | I | | | L | L | | | Q | I | L | | I | I | |
| | M | M | | | M | | | M | M | | | | L | M | | M | L | |
| | F | F | | | F | | | F | F | | | | M | F | | F | M | |
| | W | W | | | W | | | W | W | | | | W | W | | W | F | |

In some embodiments, AH₁ has an amino acid sequence having a general formula from N-terminal to C-terminal: Aro-Sma-Neg-Pos-Aro-Pos-Neg-Sma-Pho-Pos-Neg-Aro-Aro-Sma-Pos-Aro-Aro-Neg, and AH₂ has an amino sequence having a general formula from N-terminal to C-terminal: Sma-Sma-Neg-Pos-Pho-Pos-Neg-Sma-Pho-Pos-Neg-Aro-Aro-Sma-Pos-Pho-Aro-Neg. In some embodiments, the sequence of the amphipathic scaffold peptide is, from N-terminus to C-terminus Aro-Sma-Neg-Pos-Aro-Pos-Neg-Sma-Pho-Pos-Neg-Aro-Aro-Sma-Pos-Aro-Aro-Neg-Pro-Sma-Sma-Neg-Pos-Pho-Pos-Neg-Sma-Pho-Pos-Neg-Aro-Aro-Sma-Pos-Pho-Aro-Neg.

In some embodiments, AH₁ and AH₂ each have at least one phenylalanine residue. Without being bound to theory, the aromatic ring of the phenylalanine residue may be used to increase the hydrophobicity of the amphipathic scaffold peptide. The presence of the aromatic ring of the phenylalanine residue is also believed to reduce steric clashes between the scaffold peptide and lateral chains of the transmembrane segments of the membrane protein. This facilitates the binding of the scaffold peptide with the membrane protein. In some embodiments, AH₁ and AH₂ have the same number of phenylalanine residues in their respective sequences. In some embodiments, AH₁ and AH₂ have different numbers of phenylalanine residues in their respective amino acid sequences. In some embodiments, AH₁ has a greater number of phenylalanine residues than AH₂. In some embodiments, the ratio of the number of phenylalanine residues in the AH₁ and AH₂ sequences is any of 5:1, 4:1, 3:1 or 2:1; or 5:2, 4:2, 3:2 or 2:2; or 5:3, 4:3, or 3:3; or 5:4 or 4:4. In some embodiments, the number of phenylalanine residues in the AH₁ sequence is greater than 2, including e.g. 3, 4 or 5. In an example embodiment, the ratio of the number of phenylalanine residues in the AH₁ and AH₂ sequences is 4:1. In an example embodiment, the ratio of the number of phenylalanine residues in the AH₁ and AH₂ sequences is 3:1. Without being bound to any theory, it is believed that the asymmetrical phenylalanine residue distribution in the AH₁ and AH₂ sequences increases the solubility of the scaffold peptide. This in turn increases reconstitution efficiency of the stabilized membrane complexes. The asymmetrical phenylalanine residue distribution is also believed to increase stability of the amphipathic scaffold peptide on the target membrane protein. The inventors believe that efficient stabilized protein complex reconstitution is determined by a proper balance between intra-peptide associations, inter-peptide and micelle associations, peptide affinity for lipids and proteins, buffer conditions, temperature, the type of solubilizing agent, and the rate of solubilizing agent removal.

In some embodiments, the amino acid sequence of each of AH₁ and AH₂ is based on, but is not limited to an amino acid sequence derived from the class A amphipathic helixes contained in apolipoprotein A1 (apoA-1). In some embodiments, AH₁ and AH₂ each have an amino acid sequence of an apoA-1 mimetic peptide. In some embodiments, AH₁ and AH₂ each comprise an amino acid sequence of the apoA-1 mimetic peptide, 18A. In some embodiments, AH₁ and AH₂ each comprise a variant of the amino acid sequence of the apoA-1 mimetic peptide, 18A.

In some embodiments, the amphipathic scaffold peptide comprises an amino acid sequence as set forth in SEQ ID NO: 1. The sequence of SEQ ID NO: 1 is Phe-Ala-Glu-Lys-Leu-Lys-Glu-Ala-Val-Lys-Asp-Tyr-Phe-Ala-Lys-Leu-Trp-Asp-Pro-Ala-Ala-Glu-Lys-Leu-Lys-Glu-Ala-Val-Lys-Asp-Tyr-Phe-Ala-Lys-Leu-Trp- Asp.

In some embodiments, the amphipathic scaffold peptide has an amino acid sequence as set forth in SEQ ID NO: 2. The sequence of SEQ ID NO: 2 has two phenylalanine amino acid substitutions at amino acid position numbers 5 and 16 along the sequence of SEQ ID NO: 1 when counted from the N-terminus. SEQ ID NO: 2 is referred to herein as the NSPᵣ sequence. The sequence of SEQ ID NO: 2 is, from N-terminal to C-terminal, Phe-Ala-Glu-Lys-Phe-Lys-Glu-Ala-Val-Lys-Asp-Tyr-Phe-Ala-Lys-Phe-Trp-Asp-Pro-Ala-Ala-Glu-Lys-Leu-Lys-Glu-Ala-Val-Lys-Asp-Tyr-Phe-Ala-Lys-Leu-Trp-Asp.

In some embodiments, the amphipathic scaffold peptide has an amino acid sequence that is a conservative variant of SEQ ID NO: 2. In some embodiments, the amphipathic scaffold peptide has an amino acid sequence that is a conservative variant of SEQ ID NO: 1.

In some embodiments, the amphipathic scaffold peptide has an amino acid sequence having at least about 80% sequence identity to the sequences as set forth in SEQ ID NO: 1 and SEQ ID NO: 2, including any higher degree of similarity e.g. at least 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, 99.7% or 99.9% sequence similarity.

In some embodiments, the amphipathic scaffold peptide is present with a purity of at least 85%, including any higher value e.g. 86%, 87%, 88%, 89%, 90%, 91%, 92%, 93%, 94%, 95%, 96%, 97%, 98% or 99%. In some embodiments, due to the inefficiency of peptide synthesis, small quantities of shorter-chain amphipathic scaffold peptides (i.e. incomplete synthetic products) can be present and the amphipathic scaffold peptides can still be used to successfully solubilize membrane proteins.

With reference to FIG. 3, an example embodiment of the putative structure of an exemplary amphipathic scaffold peptide 26 having the amino acid sequence of SEQ ID NO:2, showing the AH₁, AH₂ and linker 30 regions is illustrated schematically. The amino acid side chains are illustrated in their putative orientations relative to the alpha-helical backbone of the AH₁ and AH₂ regions of the peptide.

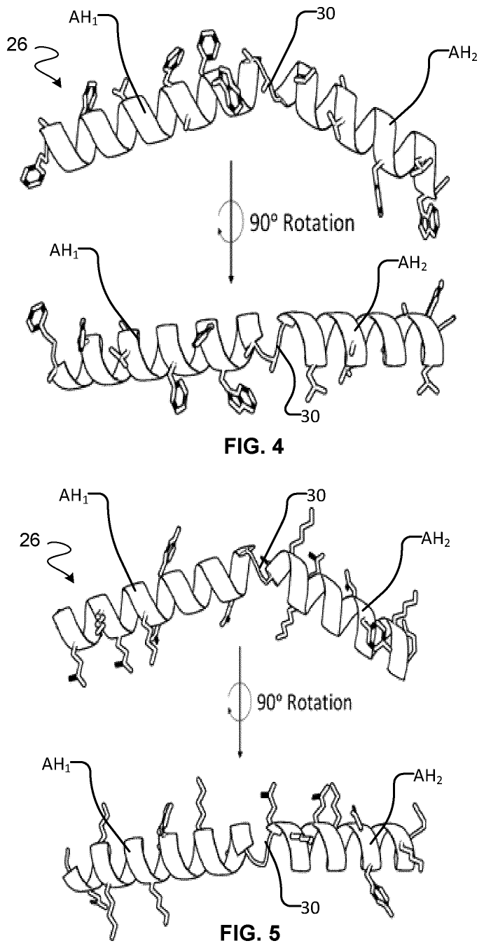

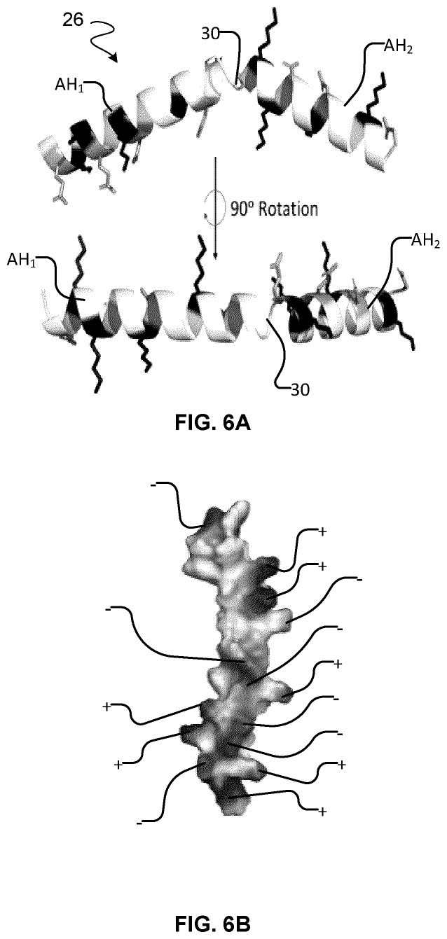

FIG. 4 shows the embodiment of FIG. 3, but with only the side chains of the hydrophobic amino acids (Leu, Ala, Phe and Val) illustrated schematically. FIG. 5 shows the embodiment of FIG. 3, but with only the side chains of the hydrophilic amino acids (Tyr, Trp, Lys, Asp, Glu) illustrated schematically. FIG. 6A shows the location of the charged amino acid residues on the amphipathic scaffold peptide of FIG. 3, wherein black indicates a positive charge, grey indicates a negative charge, and white indicates a neutral charge. FIG. 6B shows the corresponding expected location of correspondingly charged regions on a model of the structure of the amphipathic scaffold peptide.

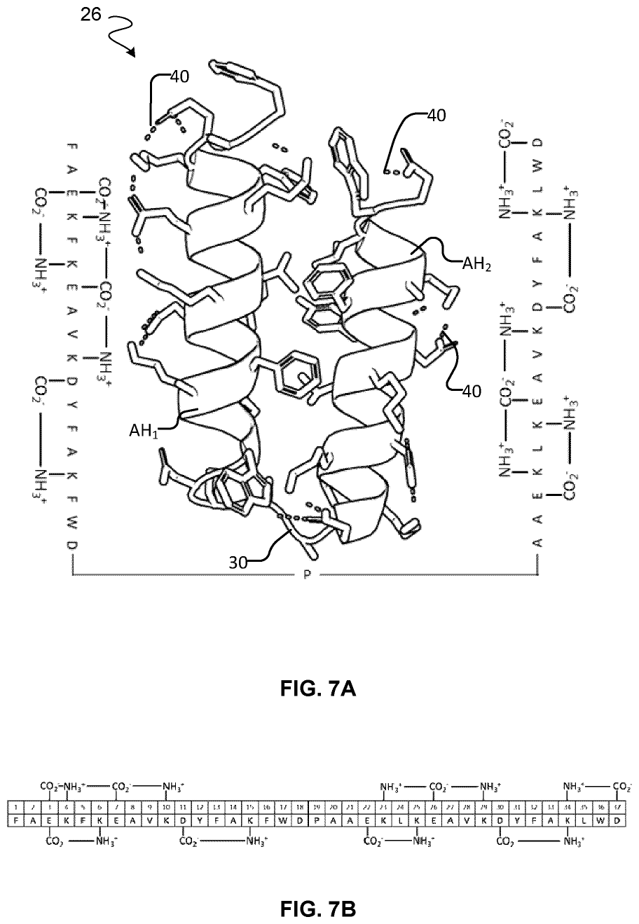

FIGS. 7A and 7B (collectively, FIG. 7) illustrate an example embodiment of an amphipathic scaffold peptide showing the locations of putative intra-helical salt bridges believed to form between charged amino acid residues in the peptide in each of AH₁ and AH₂. The amino acid sequence of the peptide according to an example embodiment having SEQ ID NO: 2 is also shown, indicating specific locations of salt bridge formations within the amino acid sequence of the scaffold peptide according to an example embodiment. The presence of the salt bridges is believed to facilitate inter-helical peptide contact such as the binding of the AH₁ region to the AH₂ region, as well as facilitating intra-helical contacts of the AH₁ and AH₂ regions, respectively, to further stabilize the amphipathic scaffold peptides. The presence of the salt bridges is also believed to facilitate scaffold peptide solubility.

In some embodiments, the total number of salt bridges intra-helically formed within each of the AH₁ and AH₂ regions is 10 or more, e.g. 11, 12, 13 or 14. In some embodiments, the total number of salt bridges intra-helically formed within each of the AH₁ and AH₂ regions is greater than 8, including e.g. 9, 10, 11, or 12. In some embodiments, the number of salt bridges intra-helically formed within each of the AH₁ and AH₂ regions can be any one of 5, 4, 3, 2 or 1.

A charged amino acid within the amphipathic scaffold peptide may form one or more salt bridges with any suitably proximate oppositely charged amino acid. Table 3 below lists example locations within the AH₁ and/or AH₂ regions at which salt bridges may be intra-helically formed, counting from the N-terminal residue of the consensus sequence of the NSPᵣ peptide.

TABLE 3
Example Locations of Salt Bridges
Within the AH₁ and the AH₂ Regions.

| | Residue Number | Possible |
|---|---|---|
| | Counting from the N- | Salt |
| | terminus of the | Bridge |
| | Peptide | Formation |
| AH₁ | 3 | 4, 6 |
| | 4 | 3, 7 |
| | 6 | 3 |
| | 7 | 4, 10 |
| | 10 | 7 |
| | 11 | 15 |
| | 15 | 11 |
| AH₂ | 22 | 25 |
| | 23 | 26 |
| | 25 | 22 |
| | 26 | 23, 29 |
| | 29 | 26 |
| | 30 | 34 |
| | 34 | 30, 37 |
| | 37 | 34 |

In an example embodiment, one or more salt bridges are formed between any one or more of: residue numbers 3 and 4, residue numbers 3 and 6, residue numbers 4 and 7, residue numbers 7 and 10, residue numbers 11 and 15 counting from the N-terminus of the NSPᵣ peptide. In an example embodiment, the side chains of the amino acids at residue numbers 3, 4, 7 and 10 are positioned at the same side of the peptide backbone. In an example embodiment, the side chains of the amino acids at residue numbers 3, 6, 11 and 15 are positioned at the same side of the peptide backbone.

In an example embodiment, one or more salt bridges are formed between any one or more of: residue numbers 22 and 25, residue numbers 23 and 26, residue numbers 26 and 29, residue numbers 30 and 34 and residue numbers 34 and 37 counting from the N-terminus of the NSPᵣ peptide. In an example embodiment, the side chains of the amino acids at residue numbers 23, 26, 29, 34 and 37 are positioned at the same side of the peptide backbone. In an example embodiment, the side chains of the amino acids at residue numbers 22, 25, 30 and 34 are positioned at the same side of the peptide backbone.

In some embodiments, one or more positively charged amino acids may each be substituted with any negatively charged amino acid and a corresponding negatively charged amino acid with which the one or more positively charged amino acids forms a salt bridge may be substituted with any positively charged amino acid. In some embodiments, one or more negatively charged amino acids may each be substituted with any positively charged amino acid, and a corresponding positively charged amino acid with which the one or more negatively charged amino acids forms a salt bridge may be substituted with any negatively charged amino acid. The swapping of corresponding pairs of positively and negatively charged amino acids and vice versa can be done as long as the corresponding salt bridges can still be formed. In some embodiments, the total number of salt bridges formed in each of the AH₁ and AH₂ regions is greater than 8, e.g., 9, 10, 11, 12, 13, 14, 15, 16, 17, 18, 19, or 20.

In some embodiments, intermolecular salt bridges may be formed between oppositely charged amino acid residues of molecules of the amphipathic scaffold peptide that are disposed proximate to one another on the surface of the hydrophobic membrane protein. In some embodiments, the number of salt bridges formed between the AH₁ and AH₂ sequences is greater than about 40, e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80. In some embodiments, the number of salt bridges formed between the AH₁ and AH₂ sequences is between 45 and 70, including any subrange therebetween.

In some embodiments, the change in Gibbs free energy (ΔG) (i.e., an indicator of the stability of the three dimensional structure of a peptide) of the amphipathic scaffold peptide is at least 29 kcal/mol, e.g., 29.1, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0 kcal/mol.

In some embodiments, the change in Gibbs free energy (ΔG) of the AH₁ region is at least 12 kcal/mol, e.g., 12.0, 12.5, 13.0, 13.5, 14.0, 14.5, 15.0, 15.5, 16.0, 16.5, 17.0, 17.5, 18.0, 18.5, 19.0, 19.5, 20, 20.5, 21.0, 21.5, 22.0, 22.5, 23.0, 23.5, 24.0, 24.5, 25.0, 25.5, 26.0, 26.5, 27.0, 27.5, 28.0 or 28.5 kcal/mol. In some embodiments, the change in Gibbs free energy (ΔG) of the AH₁ region is less than about 20 kcal/mol, e.g.,19.5, 19.0, 18.5, 18.0, 17.5, 17.0, 16.5, 16.0, 15.5, 15.0, 14.5, 14.0, 13.5, 13.0, 12.5, 12.0, 11.5, 11.0, 10.5, 10.0, 9.5, 9.0, 8.5, 8.0, 7.5, 7.0 kcal/mol. In some embodiments, the change in Gibbs free energy (ΔG) of the AH₁ region is between 12 and 20 kcal/mol including any value therebetween, e.g., 12.5, 13.0, 13.5, 14.0, 14.5, 15.0, 15.5, 16.0, 16.5, 17.0, 17.5, 18.0, 18.5, 19.0 or 19.5.

In some embodiments, the change in Gibbs free energy (ΔG) of the AH₂ region is at least 16 kcal/mol, e.g., 16.0, 16.5, 17.0, 17.5, 18.0, 18.5, 19.0, 19.5, 20, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0 kcal/mol. In some embodiments, the change in Gibbs free energy (ΔG) of the AH₂ region is greater than 17.5 kcal/mol, e.g., 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, or 22.0 kcal/mol. In some embodiments, the change in Gibbs free energy (ΔG) of the AH₂ region is between 17.5 kcal/mol and about 21 kcal/mol, e.g., 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8 or 20.9 kcal/mol.

In some embodiments, the absolute hydrophobic moment of the amphipathic scaffold peptide is less than about 7.000 A*kT/e, e.g., 6.900, 6.800, 6.700, 6.600, 6.500, 6.400, 6.300, 6.200, 6.100 or less than about 6.000 A*kT/e, e.g., 5.900, 5.800, 5.700, 5.600, 5.500, 5.400, 5.300, 5.200, 5.100, 5.000, 4.900, 4.800, 4.700, 4.600, 4.500, 4.400, 4.300, 4.200, 4.100, 4.000, 3.900, 3.800, 3.700, 3.600, 3.500, 3.400, 3.300, 3.200, 3.100, 3.000, 2.900, 2.800, 2.700, 2.600, 2.500, 2.400, 2.300, 2.200, 2.100 or 2.000 A*kT/e. In an example embodiments, the absolute hydrophobic moment of the amphipathic scaffold peptide is about 5.792 A*kT/e.

In some embodiments, the hydrophobic moment of the amphipathic scaffold peptide is at least about 20.05 kcal/mol, e.g., 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5 or 30.0 kcal/mol. In some embodiments, the hydrophobic moment of the AH₁ region is at least about 19.60 kcal/mol, e.g., 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5 or 30.0 kcal/mol. In some embodiments, the hydrophobic moment of the AH₂ region is less than 21 kcal/mol, e.g., 20.5, 20.0, 19.5, 19.0, 18.5, 18.0, 17.5, 17.0, 16.5, 16.0, 15.5 or 15.0.

In some embodiments, the absolute surface electropotential of the amphipathic scaffold peptide is between about 4.500 kT/e and about 5.000 kT/e, e.g., 4.600, 4.700, 4.800 or 4.900 kT/e. In some embodiments, the absolute surface electropotential of the amphipathic scaffold peptide is greater than about 5.100 kT/e, e.g., 5.200, 5.300, 5.400, 5.500, 5.600, 5.700, 5.800, 5.900, 6.000, 6.100, 6.200, 6.300, 6.400, 6.500, 6.600, 6.700, 6.800, 6.900 or 7.000. In an example embodiment, the absolute surface electropotential of the amphipathic scaffold peptide is about 5.234 kT/e.

FIGS. 8A and 8B are helical wheel representations of the AH₁ and AH₂ regions according to an example embodiment. Non-polar amino acid residues are denoted using boxes. The helical wheel representations show an example orientation of the polar and non-polar amino acid residues about the central axes of the AH₁ and AH₂ regions.

In some embodiments, the sequence of the amphipathic scaffold peptides is selected to meet the following criteria: 1) peptide asymmetry as to NSPr or NSP (i.e. having a number of aromatic amino acids in one of AH₁ and AH₂ that is greater than the number of aromatic acids in the corresponding AH₂ or AH₁, respectively); 2) hydrophobic moment as to NSPr; 3) length of 35-38 aminoacyls with Pro or Gly at helix interface (i.e. as the linker); 4) primary sequence based on similar amino acids of hydrophobicity, charge and bulkiness using the following formula from N-terminus to C-terminus: Aro-Sma-Neg-Pos-Aro-Pos-Neg-Sma-Pho-Pos-Neg-Aro-Aro-Sma-Pos-aro-aro-neg-Pro-Sma-Sma-Neg-Pos-Pho-Pos-Neg-Sma-Pho-Pos-Neg-Aro-Aro-Sma-Pos-Pho-Aro-Neg, wherein Neg is negative, Pos is positive, Aro is aromatic, Sma is small hydrophobic, Pho is hydrophobic; and 5) primary sequence based on alternating hydrophilic and hydrophobic amino acids following formula from N-terminus to C-terminus: 2× (2Pho-2Phi-1Pho-2Phi-2Pho-2Phi-3Pho-1Phi-2Pho-1Phi) (i.e. the sequence of each of AH₁ and AH₂ from N-terminus to C-terminus) separated by Pro, wherein Pho is hydrophobic and Phi is hydrophilic.

In an example embodiment, the solubility of the amphipathic scaffold peptide is greater than about 5 mg/mL in water, including e.g. 6, 7, 8, 9, 10, 11, 12, 13, or 15 mg/mL or higher. In an example embodiment, the solubility of the amphipathic scaffold peptide is greater than about 15 mg per ml of water, including e.g. 16, 17, 18, 19, 20, 21, 22, 23, 24 or 25 mg per ml of water or higher. The inventors have found that an amphipathic scaffold peptide with a solubility of at least about 5 mg/mL in water, including about 10-15 mg per mL in water, achieves high reconstitution efficiency. While peptide constructs with lower solubilities could work, it is believed that the solubility of the amphipathic scaffold peptide impacts the reconstitution efficiency of the stabilized membrane complex. An amphipathic scaffold peptide with low solubility may self-aggregate into nanoparticles rather than bind to the membrane protein to form stabilized membrane complexes.

In some embodiments, the amphipathic scaffold peptide has an affinity tag attached to one or both of the N-terminus and the C-terminus. The affinity tag may be used to facilitate the purification and/or detection of target membrane proteins. Examples of suitable affinity tags include, but are not limited to, biotinylation-tags, histidine-tags, Strep-tags, Avi-tags, Myc-tags, V5-tags, HA-tags, Spot-tags, 1D4 tags and NE-tags, GST-tags, JS-tags, cysteine-tags, FLAG-tags, thioredoxin-tags (TRX), chitin binding protein (CBP) or maltose binding proteins (MBP), or the like.

In some embodiments, the amphipathic scaffold peptide includes fluorescent or luminescent labels coupled to one or both of the N-terminus and the C-terminus, or to any other suitable location on the peptide. Non-limiting examples of fluorescent labels include dansyl, maleimide, fluorescein isothiocyanate (FITC), ortho-aminobenzoic acid (Abz), dinitrophenol (DNP), 4-(dimethylaminoazo)benzene-4-carboxylic acid (DABCYL), 5-((2-Aminoethyl)amino)naphthalene-1-sulfonic acid (EDANS), cyanine dyes, ATTO, Alexa and fluorescein amidite (FAM).

In some embodiments, the amphipathic scaffold peptide includes an oligonucleotide label. The oligonucleotide label may be coupled at any suitable location on the peptide. The oligonucleotide label may be coupled to one or both of the N-terminus and the C-terminus. The oligonucleotide label may be coupled to a cysteine residue.

In some embodiments, the amphipathic scaffold peptide comprises a cysteine residue at any desired location, whether N-terminal, C-terminal, or internal. In some embodiments such cysteine residue is used for thiol-maleimide modification.

In some embodiments, the amphipathic scaffold peptide may be further modified. Examples of modifications include, but are not limited to, acylation (e.g., O-acylation (esters), N-acylation (amides), S-acylation (thioesters), acetylation, formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein), polysialylation (e.g., the addition of polysialic acid), glypiation (e.g., glycosylphosphatidylinositol (GPI) anchor formation, hydroxylation, iodination, and phosphorylation (e.g., the addition of a phosphate group).

Some embodiments of the invention relate to methods for the isolation or purification of hydrophobic biomolecules using a scaffold protein. The methods for the isolation or purification of hydrophobic biomolecules using a scaffold protein may be performed using any one of size-exclusion chromatography, gel electrophoresis, affinity chromatography, density gradient centrifugation, or by removing unbound/non-incorporated lipids and/or peptides and/or detergent by: dialysis, detergent-binding biobead separation, magnetic bead separation, concentrators, or membrane filtration.

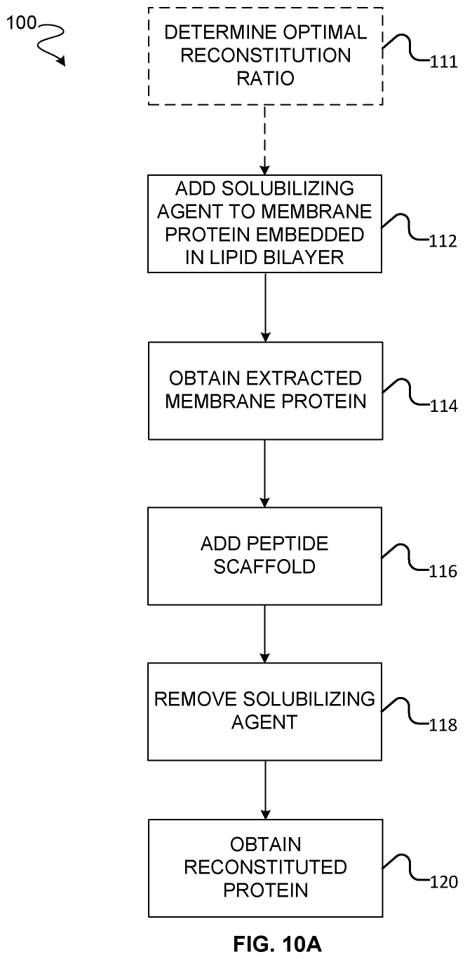

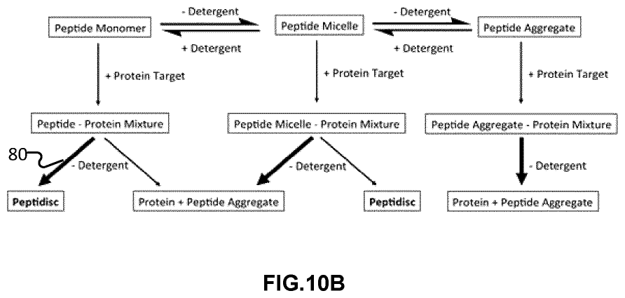

FIG. 10A is flowchart of a method according to an example embodiment of the invention. Method 100 optionally includes a step 111 of determining the optimal reconstitution ratio of amphipathic scaffold peptides to membrane protein prior to carrying out the remaining steps of method 100. At step 112, a solubilizing agent is added to an aqueous solution of a target hydrophobic membrane protein that is embedded in a lipid bilayer. At step 114, the addition of the solubilizing agent extracts the membrane protein from the lipid bilayer to yield an extracted membrane protein.

Method 100 further includes step 116 of adding a plurality of amphipathic scaffold peptides into the aqueous solution, and step 118 of removing the solubilizing agent from the aqueous solution. Steps 116 and 118 may be collectively referred to as the “reconstitution” steps. The removal of solubilizing agent from the aqueous solution in step 118 allows the plurality of amphipathic scaffold peptides to self-assemble around the hydrophobic regions of the membrane protein to form a reconstituted membrane protein, which is referred to herein as a “peptidisc” 20. In some embodiments, the reconstituted membrane protein is free of detergent. In some embodiments, the reconstituted membrane protein is free of exogenous lipids. In some embodiments, the solution containing the reconstituted membrane protein is an aqueous solution of just the membrane protein and the amphipathic scaffold peptides.

FIG. 10B shows the hypothetical efficacy of membrane protein reconstitution using an amphipathic scaffold peptide according to an example embodiment. The thickness of the lines corresponds with the extent of favourability of a reaction. It is hypothesized that the use of an amphipathic scaffold peptide according to an example embodiment drives the formation of scaffold peptide:membrane protein complex (“peptidisc”) in the absence of detergent, as indicated by heavy arrow 80. The inventors believe that efficient membrane protein reconstitution is determined by the proper balance between factors such as intra-peptide associations, inter-peptide associations and micelle associations, peptide affinity for lipids and proteins, and other experimental conditions such as the types of buffer and detergent used, the operating temperature and the rate of detergent removal. Without being bound by theory, it is believed that asymmetrical bi-helical scaffold peptides have a moderate intra-peptide association compared to symmetrical bi-helical scaffold peptides. Micelles of asymmetrical bi-helical scaffold peptides stabilize more easily and maintain their solubility at a lower detergent concentration.

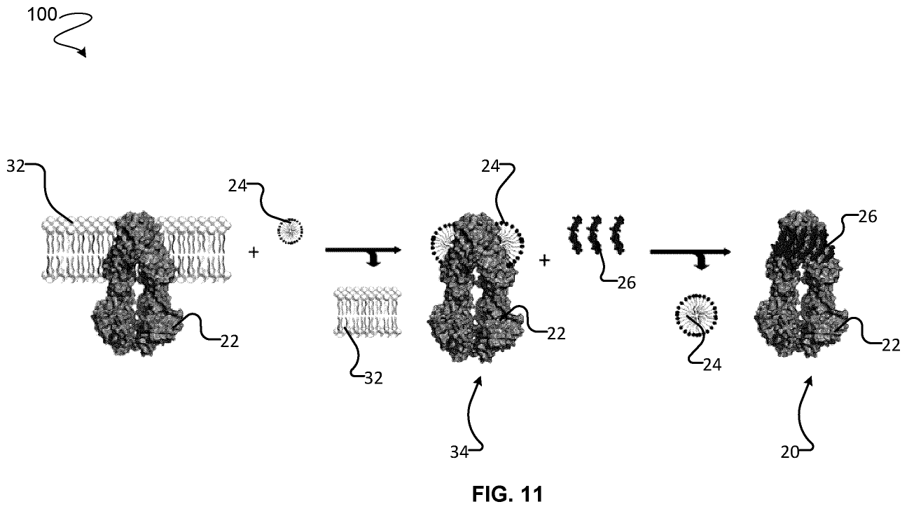

With reference to FIG. 11, a view of the effects of carrying out method 100 at the molecular level is illustrated schematically. First, a membrane protein 22 in a lipid bilayer 32 is obtained. A solubilizing agent such as detergent 24 is added to separate the membrane protein 22 from the lipid bilayer 32, yielding an extracted membrane protein 34 in an aqueous solution.

Next, a plurality of amphipathic scaffold peptides 26 are added to the aqueous solution, and the detergent 24 or other solubilizing agent is removed. The plurality of amphipathic scaffold peptides 26 self-assemble around the membrane protein 22 to yield a reconstituted membrane protein, referred to herein as a “peptidisc” 20. When assembled, the amphipathic scaffold peptide formed from peptides 26 acts to shield the membrane protein 22 from the aqueous solution, thereby solubilizing the membrane protein 22. Without being bound by theory, it is predicted that the multiple copies of scaffold peptide 22 contact the hydrophobic regions of membrane protein 16 or the alkyl chains of annular lipids (i.e., the lipids which preferentially bind to the surface of membrane proteins) by orienting themselves onto membrane protein 22 in a tilted manner (as best shown in FIG. 2C). In some embodiments, small amounts of lipids are optionally added to membrane protein 22 with the plurality of amphipathic scaffold peptides 26 during the reconstitution step.

In some embodiments, method 100 optionally includes step 111 of determining an optimal membrane protein to scaffold peptide ratio for use in the reconstitution method. The optimal reconstitution ratio varies depending on the identity of the target membrane protein. For example, the optimal reconstitution ratio may depend on the diameter of the hydrophobic region of the target membrane protein. For example, in some embodiments the amount of scaffold peptide required for reconstitution may increase as the diameter of the hydrophobic region of the target membrane protein increases. Step 111 may be performed before step 112. In some embodiments, the optimal reconstitution membrane protein:scaffold peptide ratio is determined by performing polyacrylamide gel electrophoresis (“PAGE”), specifically Native PAGE, or “non-denaturing” gel electrophoresis. This method is hereinafter referred to as the “on-gel” method.

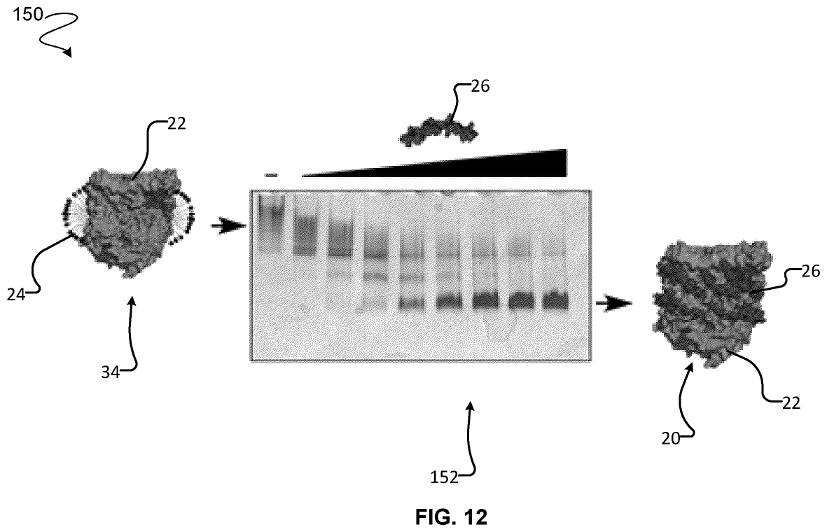

FIG. 12 is a schematic diagram illustrating a method of determining an optimal reconstitution membrane protein:scaffold peptide ratio according to an example embodiment of the invention. Method 150 comprises preparing a plurality of samples by combining a fixed known concentration of detergent purified membrane protein target 34 with increasing concentrations of amphipathic scaffold peptides 26. The samples are then loaded on a native-gel 152 to run the electrophoresis. When the electrophoresis is complete, native-gel 152 is stained with a dye. The stained gel would allow for the visual determination of the amount of peptidisc 20 and the level of purity of peptidisc 20 that is generated from each combination of detergent extracted membrane protein target 34 and amphipathic scaffold peptide 26. It is believed that the optimal reconstitution ratio would be one at which the target membrane protein does not aggregate at the top of the gel (e.g. left-most columns of native-gel 152, but instead migrates in a soluble form to its expected molecular weight position, e.g. as in the columns on the right-hand side of native-gel 152.

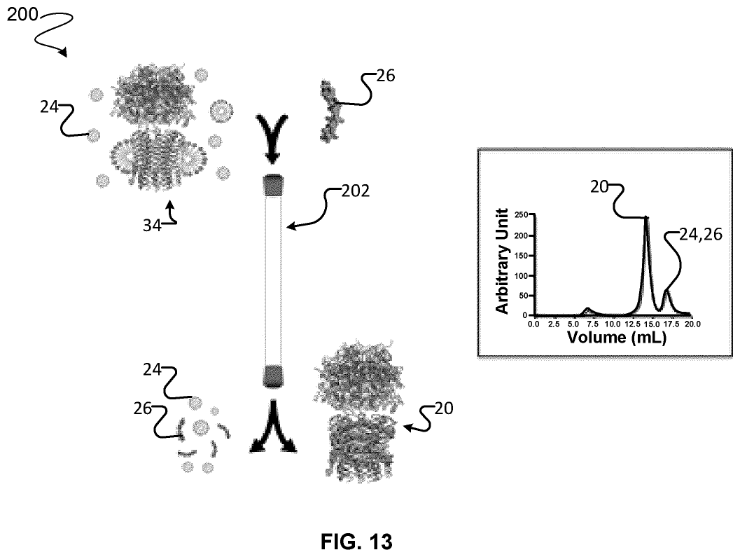

In some embodiments, the reconstitution steps 118 and 120 of method 100 are performed using size exclusion chromatography. FIG. 13 is a schematic diagram illustrating a reconstitution method of forming peptidiscs using size exclusion chromatography according to an example embodiment. Reconstitution method 200 may be referred to as the “on-column” reconstitution method. Method 200 comprises combining detergent extracted membrane protein target 34 and amphipathic scaffold peptide 26, and then loading into a size exclusion column 202. The peptidiscs 20, solubilizing agent 24, and excess peptides 26 are separated by differences in size as they pass through column 202, as shown by the inset panel of FIG. 13.

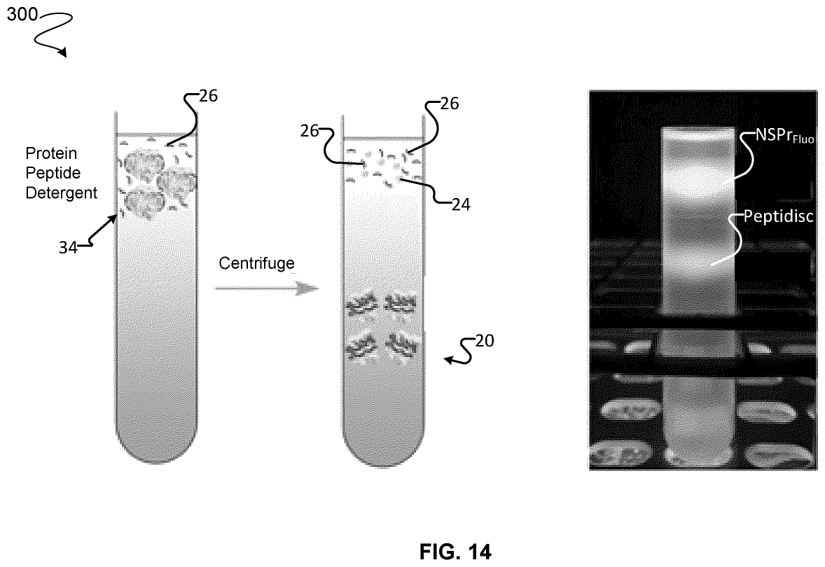

In some embodiments, the reconstitution steps 118 and 20 are performed using a density gradient centrifugation method. An example is the sucrose density gradient centrifugation method. FIG. 14 is a schematic diagram illustrating a reconstitution method of forming peptidiscs using density gradient centrifugation according to an example embodiment. Reconstitution method 300 may be referred to as the “on-gradient” reconstitution method. Method 300 comprises combining detergent extracted membrane protein 34 and amphipathic scaffold peptide 26, and then loading onto a gradient such as a linear sucrose gradient. Peptidiscs 20, solubilizing agent 24, and excess amphipathic scaffold peptides 26 are separated according to density by centrifugation, e.g. as shown schematically in the middle panel of FIG. 14, and as shown experimentally using fluorescently labelled amphipathic scaffold peptides 26 on the right-hand panel of FIG. 14.

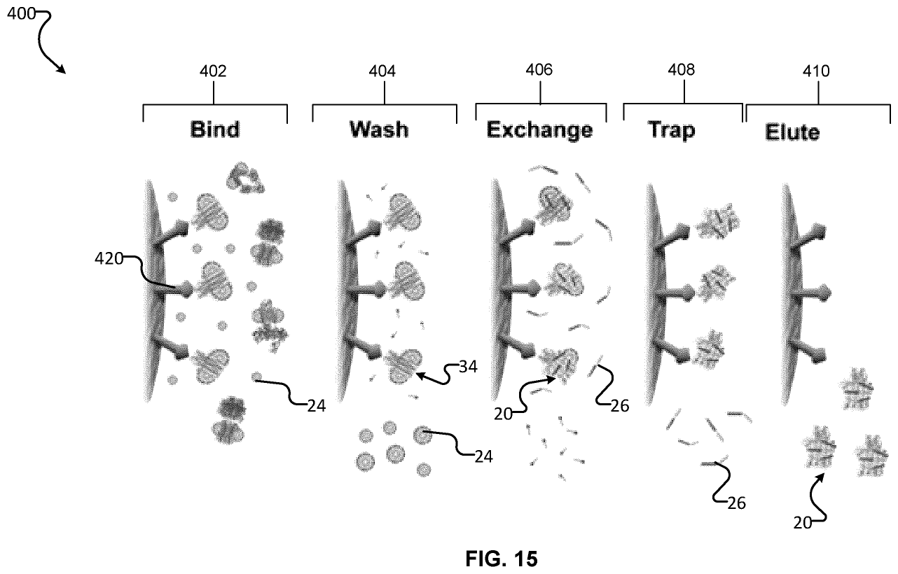

In some embodiments, the reconstitution steps 118 and 120 are performed using affinity chromatography. FIG. 15 is a schematic diagram illustrating a reconstitution method of forming peptidiscs using affinity chromatography according to an example embodiment. Reconstitution method 400 may be referred to as the “on beads” reconstitution method. Method 400 comprises step 402 of binding detergent extracted membrane protein 34 onto an affinity resin 420. Any suitable affinity resin may be used. An example is the nickel-nitrilotriacetic acid (Ni-NTA) affinity resin.

Method 400 further comprises step 404 of washing away impurities such as excess solubilizing agent 24 and other membrane proteins. In step 406, amphipathic scaffold peptides 26 are added to the solution that contacts the affinity resin. Amphipathic scaffold peptides 26 bind to target membrane protein 22, resulting in the formation of peptidiscs 20. Solubilizing agent 24 dissociates from target membrane protein 22 once amphipathic scaffold peptides 26 bind to target hydrophobic membrane protein 22. In step 408, excess amphipathic scaffold peptides 26 are washed away from affinity resin 420 while peptidiscs 20 remain trapped on the affinity resin 420, and step 410 involves eluting out the purified peptidiscs 24, for example by adding imidazole or histidine where the affinity resin is an Ni-NTA resin.

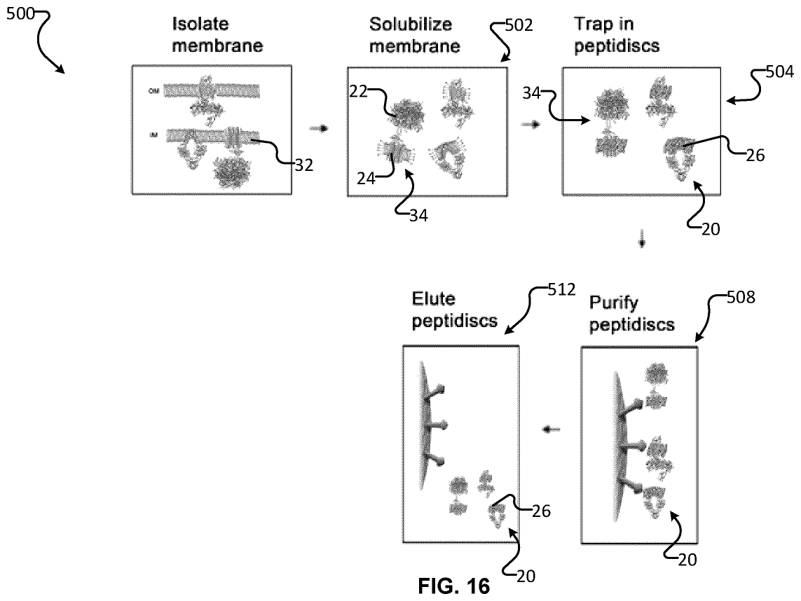

In some embodiments, peptidiscs are prepared using affinity chromatography involving N-terminal or C-terminal affinity-tagged scaffold peptides. FIG. 16 is a schematic diagram illustrating a method 500 of forming peptidiscs using affinity chromatography according to an example embodiment. Method 500 comprises step 502 of adding solubilizing agent 24 to extract membrane protein 22 from lipid bilayer 32 to create an extracted membrane protein 34. Step 504 involves incubating extracted membrane protein 34 with affinity-tagged amphipathic scaffold peptides 26, and step 506 involves reconstituting into peptidiscs 20 by using any suitable reconstitution method. Method 500 further comprises step 508 of binding affinity-tagged peptidiscs 20 on an affinity resin. Contaminants, residual solubilizing agent 24, and excess amphipathic scaffold peptides 26 (not shown) are washed out in step 510 so that purified peptidiscs 20 are then eluted out in detergent-free buffer in step 512.

In some embodiments, a method for incorporating a hydrophobic membrane protein into a nanoscale peptidisc particle which is stable and soluble in aqueous solution is provided. The method includes steps of (a) using a solubilized hydrophobic protein of interest and a solubilizing agent in an aqueous solution; (b) using a nano scaffold peptide with the solubilized membrane protein (a) and (c) methods to remove the solubilizing agent so that the scaffold peptide self-assemble into nanoscale peptidisc particles in an aqueous solution.

Some embodiments of the invention relate to a method of using an amphipathic scaffold peptide comprising any one of the above amino acid sequences to purify membrane proteins without the use of detergents. The amphipathic scaffold peptide may be used as a research tool to allow for the study of membrane proteins of different size, topography, and complexity.

Amphipathic scaffold peptides can be used to stabilize membrane proteins with different structures. Membrane proteins that can be stabilized using the amphipathic scaffold peptide according to some embodiments include proteins having transmembrane α-helices, β-sheets, turns, and combinations thereof, and lipid-modified proteins, lipid-protein complexes, chimeric proteins with fused hydrophobic and/or transmembrane domains, lipid-based nanoparticles, and peripherally-bound membrane proteins and membrane complexes thereof.

Non-limiting examples of methods of use of the amphipathic scaffold peptide are as follows:
Structural and functional studies of membrane proteins.
Studying membrane protein interactions with another membrane protein or soluble protein partner.
Trapping of membrane proteins during their purification.
Trapping of membrane protein following in vitro synthesis of same.
NMR and surface binding studies (e.g., surface plasmon resonance (SPR), biolayer interferometry (BLI) and microscale thermophoresis (MST) analysis) of membrane proteins.
Drug delivery, including via stabilization of lipid-based nanoparticles containing hydrophobic drug payloads to facilitate drug delivery.
Drug testing and drug discovery targeted at membrane proteins.
Developing and selecting antibodies for surface labeling and therapeutic use.
Cataloguing membrane proteome and interactome.
Finding candidate binding partners for proteins of interest.
Mass spectrometry on intact membrane proteins.
Establishing peptidisc membrane protein libraries.
Immunogen for antibody generation using animal immunization.
Immunogen for antibody generation using display technologies.
Vaccination formulations.
Cosmetic formulations.

Examples of some proteins that have been stabilized and purified using the amphipathic scaffold peptides according to some embodiments include those listed in Table 4, including alpha-helical membrane proteins, beta-barrel membrane proteins, transmembrane proteins having between 4 and 36 transmembrane segments (TMS) (including any value therebetween e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 transmembrane segments), monomeric membrane proteins and oligomeric membrane proteins.

In some embodiments, amphipathic scaffold peptides are used to stabilize various membrane proteins including transporters, lipid flippases, porins, channels, ligand-gated channels, G-protein coupled receptors (GPCRs), large membrane protein assemblies, and the like.

TABLE 4
Exemplary Membrane Proteins that can be Stabilized Using
Amphipathic Scaffold Peptides in Some Embodiments

| Type | Name | Function | Source |
|---|---|---|---|
| ABC Transporters | ABCG5/G8 | Strerol transport | P. pastoris |
| | MsbA | Lipid A export | E. coli |
| | MalFGK₂ | Maltose importer | |
| Lipid Flippases | ATP8A2 | P4-Lipid flippase | Expi293 |
| | ABCA4 | Transretinal lipid | HEK293 |
| | | flippase | |
| Porins | FhuA | Iron uptake across | E. coli |
| | | outer membrane | |
| | BtuB | Vitamin B12 receptor | |
| | OmpF₃ | Ion Porin trimer | |
| Channels | SecYEG | Protein translocon for | |
| | | protein excretion | |
| | MscS | 7-subunit mechano- | |
| | | sensitive channel | |
| | NaV | Voltage-gated sodium | |
| | | channel | |
| Peripheral and | BamABCDE | Outer membrane | HEK293 |
| lipid anchored | | assembly machine | |
| membrane proteins | | | |
| GPCRs | CB1 | Cannabinoid receptor | HEK293 |
| | μOpioid | μOpiod receptor | Insect sf9 |
| | Receptor | | Cells |
| Ligand Gated | 5HT3 | Serotonin receptor | HEK293 |
| Channel | | | |
| Large Membrane | BRC | Photosynthetic bacterial | E. coli |
| Protein Assembly | | reaction centre | |

### EXAMPLES

Embodiments of the invention are further described with reference to the following examples, which are illustrative and not limiting in nature.

### Materials and Methods

Preparation of the amphipathic peptides: For solubility test experiments, lyophilized NSP having SEQ ID NO: 11 and NSPᵣ having SEQ ID NO: 2 (purity of 82% and 85%, respectively) were resuspended in dH₂0 at room temperature to final concentrations of 15 mg/mL and 25 mg/mL, respectively. Peptide concentration was determined by absorbance at 280 nm. Residual trifluoroacetic acid (TFA) from peptide synthesis results in a low pH solution (pH 2-3). For all other experiments, peptides were solubilized in dH₂O at 6 mg/mL. Solubilized peptides were stored at 4° C. for up to 5 weeks. Immediately before use, the pH of the peptide solution was modified by addition of 20 mM Tris-HCl, pH 8 to form the so-called Assembly Buffer. Immediately before use in peptidisc reconstitutions, peptide concentration in Assembly Buffer was verified by Bradford assay.

Protein expression and purification: Unless otherwise stated, all proteins were expressed in E. coli BL21(DE3) (New England Biolabs) for 3 hours at 37° C. after induction at an OD of 0.4-0.7 in Luria-Bertani (LB) medium supplemented with required antibiotic. Cells were harvested by low speed centrifugation (10,000×g, 6 min) and resuspended in Buffer A (50 mM Tris-HCl: pH 8; 100 mM NaCl; 10% glycerol). Resuspended cells were treated with 1 mM phenylmethylsulfonyl fluoride (PMSF) and lysed using a microfluidizer (Microfluidics) at 10,000 psi. Unbroken cell debris and other aggregates were removed by an additional low speed centrifugation. Cytosolic and crude membrane fractions containing the overexpressed protein of interest were subsequently isolated by ultracentrifugation (100,000×g, 45 minutes) and crude membrane fraction resuspended in Buffer A (50 mM Tris-HCl: pH 8, 100 mM NaCl, 10% glycerol). MalE and His-tagged MalFGK₂ were purified as previously described, (H. Bao, F. Duong, PLoS One 2012, 7(4), e34836.) expressed from plasmids pBAD33-MalE and pBAD22-FGKₕᵢₛ, respectively. Crude membranes containing His-tagged MalFGK₂ were solubilized at 4° C. overnight in Buffer A+1% DDM and clarified by ultracentrifugation.

Solubilized MalFGK₂ was isolated by Ni²⁺-chelating chromatography in Buffer A+0.02% DDM, washed in 5 column volumes (CV) of Buffer B (50 mM Tris-HCl: pH 8; 200 mM NaCl; 15 mM imidazole; 10% glycerol)+0.02% DDM, and then eluted in Buffer C (50 mM Tris-HCl: pH 8; 100 mM NaCl; 400 mM imidazole; 10% glycerol)+0.02% DDM. Protein MalE was isolated on Resource 15Q column, concentrated using a 30 kDa polysulfone filter (Pall Corporation), and then further purified on Superdex™ 200HR 10/300 GL column equilibrated in Buffer EQ (50 mM Tris-HCl: pH 8, 50 mM NaCl, 10% glycerol). His-tagged-MSP_{L156} and His-tagged TonB₂₃₋₃₂₉ were purified by Ni²⁺-chelating chromatography as previously described (A. Mills, H. T. Le, J. W. Coulton, F. Duong, Biochim. Biophys. Acta-Biomembr. 2014, 1838.). His-tagged Colicin M was expressed and purified according to established protocols from plasmid pMLD189 in the E. coli strain BW25113 (A. Mills et al., 2014). His-tagged FhuA, encoded by plasmid pHX405, was expressed in E. coli strain AW740 (ΔompF, ΔompC) in M9 minimal media, and was purified in lauryl-dimethylamine-N-oxide (LDAO) as previously described (A. Mills et al., 2014). OmpF was expressed from E. coli JW2203 (ΔOmpC) as previously described (D. Jun, R. G. Saer, J. D. Madden, J. T. Beatty. Photosynth. Res. 2014, 120 (1-2), 197-205.). Prepared crude membrane was resuspended in Buffer A and the inner membrane solubilized by addition of 1% Triton™ X-100. The outer membrane fraction (OM) was isolated by ultracentrifugation, resuspended in Buffer A+1% LDAO at a concentration of 3 mg/mL, and incubated overnight at 4° C. with gentle rocking. Insoluble material was removed by an additional ultracentrifugation step, and the clarified lysate was applied onto a Resource 15Q column pre-equilibrated in Buffer EQ+0.1% LDAO. OmpF was eluted by a linear 20 mL gradient of 50-700 mM NaCl, and further purified by Superdex™ 200HR 10/300 in Buffer A+0.1% LDAO. Expression and purification of His-tagged SecYEG was performed from the plasmid pBad22-His-EYG as previously described (K. Dalai, C. S. Chan, S. G. Sligar, F. Duong, Proc. Natl. Acad. Sci. U.S.A. 2012, 109(11).). Crude membranes were solubilized for one hour at 4° C. in Buffer A+1% n-dodecyl-b-D-maltopyranoside (DDM). Solubilized material was clarified by ultra-centrifugation and passed over a 5 mL Ni²⁺-NTA column. After extensive washing in Buffer A+0.02% DDM, SecYEG was eluted in Buffer A+0.02% DDM over a 20 mL gradient of 0-600 mM imidazole. The most concentrated fractions were pooled and diluted 5-fold in Buffer O (50 mM Tris-HCl: pH 8, 10% glycerol+0.02% DDM) before being applied to a 5 mL Fast Flow S cation exchange column pre-equilibrated in Buffer EQ+0.02% DDM. Bound protein was eluted over a 20 mL gradient from 50-600 mM NaCl in Buffer EQ+0.02% DDM. Plasmids pET28 encoding his-tagged MSPD1 and MSP1D1E3 proteins were transformed into BL21 cells and protein expression and purification was performed as previously described (Dalai et al., 2012). All proteins, with the exception of BRC, were flash frozen in liquid nitrogen immediately after purification and stored at −80° C. for later use. BRC was purified as previously described (M. Bradford. Anal. Biochem. 1976, 72 (1-2), 248-254.). In brief, His-tagged BRC was expressed in Rhodobacter sphaeroides RcX (ΔpuhA, ΔpufQBALMX, ΔrshI, ΔppsR) using plasmid pIND4-RC1. A preculture of 10 mL in RLB media (LB medium; 810 μM MgCl₂; 510 μM CaCl₂)+25 μg/mL kanamycin was transferred into 100 ml of RLB-kan and grown overnight at 30° C. before transfer into 1 L of freshly prepared RLB-kan. After growth for 8 hours at 30° C., BRC production was induced with 1 mM IPTG for an additional 16 hours. During growth and purification, light exposure was kept to a minimum. Cells were harvested by low speed centrifugation, resuspended in Buffer A and lysed by French press (10,000 psi). Unbroken cells and cell debris were removed by low speed centrifugation, and the supernatant treated with 1% LDAO overnight at 4° C. After removal of insoluble material by ultracentrifugation, the supernatant was supplemented with 10 mM imidazole and the BRC purified by Ni²⁺-chelating affinity chromatography. BRC bound to affinity resin was washed overnight at 4° C. in 20 column volumes of Buffer B+0.03% LDAO, before elution in Buffer C+0.03% LDAO. The complex was further purified on a Superdex 200HR 10/300 GL in Buffer A+0.03% LDAO, and stored in the dark at 4° C. before use in thermostability assays.

“On-column” peptidisc reconstitution: MalFGK₂ (300 μg) in Buffer E+0.02% DDM was mixed with NSPr (480 μg) in Assembly Buffer in a total volume of 100 μL. The mixture was immediately injected onto a 100 μL loop connected to a Superdex™ 200HR 5/200 GL column running at 0.4 ml/min in Buffer AC (50 mM Tris-HCl, pH 8; 100 mM NaCl). Fractions were collected, pooled, concentrated using a 100 kDa polysulfone filter (Pall Corporation), and stored at 4° C. For on-column reconstitution of FhuA, 500 μL of the protein (1 mg) was mixed with NSPr (1.8 mg) in Buffer A+0.05% LDAO, and injected onto a 500 μL loop connected to a Superdex™ 200HR 10/300 GL column running at 0.5 ml/min in Buffer AC.

“In-gel” peptidisc reconstitution: The target membrane protein (˜1.25 μg) was mixed with increasing concentrations of NSPᵣ (0-2.5 μg) and allowed to incubate for 1-2 minutes at room temperature. The mixture was then supplemented with Buffer A to bring the final detergent concentration below its CMC (0.008% and 0.01% for DDM and LDAO, respectively) while keeping the final volume to 15 μL. A solution of glycerol was added to 10% final to facilitate loading on 4-12% CN-PAGE. The electrophoresis was set constant at 25 mA for 1 hour at room temperature. Bands were visualized by Coomassie Blue G250 staining.

“On-bead” peptidisc reconstitution: Crude membranes (10 mL at 7.5 mg/ml total protein content) containing overexpressed MalFGK₂ were solubilized in Buffer A+1% DDM for 1 hour at 4° C. before removal of insoluble aggregate by ultracentrifugation (100,000×g, 1 hour, 4° C.). The solubilized membrane proteins were incubated with 200 μl of Ni-NTA resin (Qiagen) pre-equilibrated in Buffer A+0.02% DDM for 1 hour at 4° C. The Ni-NTA beads were collected by low-speed centrifugation (3,000×g, 3 min), washed twice with 10 CV of Buffer B supplemented with 0.02% DDM. Post-washing, 10 CV of Assembly Buffer (1 mg/mL NSPr in 20 mM Tris-HCl pH 8) was added to the beads and allowed to incubate for 5 minutes on ice. The Assembly Buffer was removed and the beads loaded into a gravity column with 10 CV of Buffer B (50 mM Tris-HCl, pH 8; 200 mM NaCl, 10% glycerol, 15 mM imidazole). The assembled peptidiscs were subsequently treated with 500 μL of Buffer C (50 mM Tris-HCl, pH 8; 100 mM NaCl; 10% glycerol; 400 mM imidazole) to elute the peptidisc from the affinity resin. The same procedure was done in parallel, except the NSPᵣ was omitted from the Assembly Buffer and 0.02% DDM was included in Buffer A, B, and C.

Reconstitution of the BRC in peptidiscs, low lipid nanodiscs, and styrene maleic acid nanoparticles: The purified BRC complex (1 mg/mL) was mixed at a 1:1.8 (μg/μg) ratio with NSPᵣ followed by 10-fold dilution in Buffer A to decrease the LDAO concentration to 0.003%. For formation of low-lipid nanodiscs, the purified BRC complex was instead mixed at a 1:2 (mol/mol) ratio with MSP1D1 before dilution. Alternatively, an equivalent amount of BRC was diluted in Buffer A supplemented with 0.03% LDAO, 0.02% DDM, 0.1% SMA or 0.1% SDS as described. After incubation for 10 minutes on ice, aggregated proteins were removed by centrifugation (13,000×g, 10 min at 4° C.). Peptidisc formation was confirmed by analysis on CN-PAGE.

Reconstitution of MalFGK₂ and BRC in proteoliposomes: Proteoliposomes were prepared at a molar protein:lipid ratio of 1:2000. Total E. coli lipids were dissolved in chloroform, dried under nitrogen and resuspended in Buffer A+0.8% β-OG. Purified MalFGK₂ was added to the solubilized lipids, and the detergent was removed by overnight incubation at 4° C. with Amberlite™ XAD-2 adsorbent beads (Supelco). The proteoliposomes were isolated by ultracentrifugation (100,000×g, 60 min at 4° C.) and resuspended in 20 mM Tris-HCl, pH 8 before use in ATPase assays. The same procedure was employed for the BRC, but a lipid mixture of dioleoylphosphatidylcholine:dioleoylphosphatidylglycerol bilayer (DOPC:DOPG) (80:20 mol/mol) was utilized in place of total E. coli lipids.

Native gel electrophoresis: Equal volumes of 4% and 12% acrylamide solutions were prepared in advance. Linear gradient gels were formed by gradual mixing of the two solutions (35 mL each) at a flow rate of 2 ml/min using a 100 mL gradient mixer (Sigma). The cross-linking agents, tetramethylethylenediamine (TEMED) and ammonium persulfate, were added immediately before gradient mixing. Once poured, plastic wells (Biorad) were inserted and gels allowed to cure for 90 minutes before storage at 4° C. For clear-native PAGE, anode and cathode buffers consisted of Buffer N (37 mM Tris-HCl; 35 mM Glycine; pH 8.8). For blue-native PAGE, anode buffer consisted of Buffer N+180 μM Coomassie Blue G-250, and cathode buffer contained Buffer N only.

Dynamic and static light scattering analysis: Aliquots of MalFGK₂-NSPr were analyzed by static light scattering. Static light scattering analysis were performed using a WTC-050S5 column (Wyatt Technologies) connected to a miniDAWN light scattering detector and interferometry refractometer (Wyatt Technologies). Data were recorded in real time and the molecular masses were calculated using the Debye fit method using the ASTRA software (Wyatt Technology).

FhuA binding assay: FhuA-MSPL156 nanodiscs were prepared as previously described. (S. C. Lee, T. J. Knowles, V. L. G. Postis, M. Jamshad, R. A. Parslow, Y. Lin, A. Goldman, P. Sridhar, M. Overduin, S. P. Muench, T. R. Dafforn, Nat. Protoc. 2016, 11 (7), 1149-1162.). FhuA-NSPᵣ was prepared by on-column peptidisc reconstitution. About 2 μg of FhuA reconstituted into either MSPL156 or NSPᵣ was incubated with TonB23-329 (2 μg) or ColM (5 μg) in the presence or absence of ferricrocin for 5 minutes at room temperature. The protein complexes were separated by CN-PAGE and visualized by Coomassie blue staining. Neither monomeric TonB nor ColM migrate on CN-PAGE due to their isoelectric points >pH 8.8.

Absorbance spectroscopy: Absorption spectra were recorded using a Hitachi U-3010 spectrophotometer. A blank measurement was recorded in Buffer A(+0.03% LDAO for detergent purified BRC). Samples were incubated in a PCR thermocycler at the indicated temperature, and then measured at the desired time points in a quartz cuvette at room temperature. Spectra were collected between 600 nm and 1100 nm (scan time 20 sec) at intervals of 1.5 min. For comparisons of spectra between conditions, spectra were normalized to a value of 1.0 at 804 nm.

Fluorescence measurements: The BRC complex into the indicated detergent or reconstituted into peptidiscs was incubated at varying temperatures in a PCR thermocycler for 5 minutes, then 3 μL of the mixture dotted onto nitrocellulose paper pre-wetted in Buffer A. The dot blot was imaged using a LICOR odyssey infrared fluorescence scanner (excitation 680 nm, emission 700 nm). Fluorescence intensity was quantified by Image J.

NSPr quantification: The MalFGK₂ and BRC peptidiscs were prepared by on-column reconstitution on a Superdex™ 5/25 column equilibrated in Buffer A, followed by one additional gel filtration step to ensure full removal of free NSPr. MalFGK2 (1 μg), FhuA (2 μg), and BRC (2 μg) peptidiscs were analyzed by 15% SDS-PAGE. Gels were stained with Coomassie Blue G-250, and destained overnight before fluorescence measurement (excitation 680 nm, emission 700 nm) on a LICOR Odyssey scanner. The band corresponding to the NSPr peptide was quantified by densitometry using Image J and compared to a standard curve of NSPr (0-2 μg) loaded on the same gel. The determined NSPr amount was then subtracted from the total amount of protein loaded on the gel to determine the amount of reconstituted membrane protein in the peptidisc. Membrane protein content in peptidisc (g)=total protein in peptidisc (g)−measured NSPr content (g). These calculated mass measurements and the molecular weight (MW) for NSPr (4.5 kDa), MalFGK2 (173 kDa), FhuA (80 kDa) and BRC (94 kDa) were used to calculate NSPr stoichiometry as follows;

$$NSPr Stoichiometry = \frac{MW Membrane protein ( g / mol )}{MW MSPr ( g / mol )} \times \frac{Measured NSPr content ( g )}{protein content in peptidisc ( g )}$$

Each experiment was repeated in triplicate on three different gels. It is noted that detergent-purified FhuA co-purified with a contaminant, thought to be short chain lipopolysaccharides, that migrated to the same position as NSPr, therefore FhuA was reconstituted using NSPᵣ labelled with a biotin group (NSPᵣbio). To quantify NSPrbio, western blots were incubated with streptavidin conjugated to Alexafluor™ 680 in phosphate buffered saline (PBS), followed by several washes in PBS+0.1% Tween. Western blots were imaged on a LICOR Odyssey scanner fluorescence (excitation 680 nm, emission 700 nm), and bands corresponding to NSPrbio quantified in Image J.

Lipid extraction and quantification: The MalFGK₂ and BRC peptidiscs were prepared on-bead, and the FhuA peptidisc was prepared on-column. MalFGK₂ (40 μg), FhuA (40 μg), and BRC (80 μg) peptidiscs were diluted to a final volume of 200 μL of Buffer A, then mixed with 800 μL of a 2:1 solution of methanol:chloroform for 10 minutes at 25° C. in glass screw cap vials. 200 μL of chloroform and 200 μL of distilled water were added sequentially, vortexed briefly, and the resulting two phase system separated by low speed centrifugation (3,000 r.p.m., 10 minutes). The organic phase was dried under nitrogen, and stored at −20° C. Total phosphate content was determined by a modified version of the malachite green assay (P. A Lanzetta, L. J. Alvarez, P. S. Reinach, O. a. Candia, Anal. Biochem. 1979, 100 (1), 95-97.). Malachite green reagent was prepared as follows: ammonium molybdate (4.2 g) was dissolved in 100 mL of 4M HCl, then mixed with 300 mL malachite green (135 mg) dissolved in distilled water. The solution was mixed for 1 hour at 4° C., filtered, and stored at 4° C. before use. Dried lipid extracts were subsequently incubated with 1 mL of 70% perchloric acid for 3 hours at 130° C., and then 20 μL of the resulting solution mixed with 500 μL of the malachite green reagent for 5 minutes at room temperature before absorbance measurement at 660 nm. Phosphate standards (KH₂PO₄) were diluted into perchloric acid and used to prepare a standard curve with phosphate concentrations ranging from 0.01 nmol to 1 nmol PO₄. For thin layer chromatography (TLC) analysis, dried lipids were resuspended in 30 μL of chloroform, and 10 μL were dotted onto a TLC Silica gel 60 (Millipore). The TLC was developed in a solution of 35:25:3:28 chloroform:triethylamine:dH₂O:ethanol. Plates were dried in an oven for 5 minutes at 150° C. Lipids were visualized by lightly wetting plates in a solution of 10% Cu₂S0₄ in 8.5% phosphoric acid, followed by heating for 5 minutes at 150° C.

Other methods: The MalFGK₂ ATPase activity was determined by monitoring the release of inorganic phosphate using the malachite green method (Lanzetta et al., 1979). Protein and peptide concentrations were determined by Bradford assay (G. Prehna, G. Zhang, X. Gong, M. Duszyk, M. Okon, L. P. Mcintosh, J. H. Weiner, N. C. Strynadka, J. Struct. Des. 2012, 20 (7), 1154-1166). SMA polymer containing 2:1 styrene to maleic acid ratio was prepared following the procedure described by Dorr et al. (J. M. Dorr, M. C. Koorengevel, M. Schafer, A. V. Prokofyev, S. Scheidelaar, E. A. W. van der Cruijsen, T. R. Dafforn, M. Baldus, J. A. Killian. Proc. Natl. Acad. Sci. U.S.A. 2014, 111). In brief, 10% of SMA 2000 (Cray Valley), was refluxed for 3 hours at 80° C. in 1M KOH, resulting in complete solubilization of the polymer. Polymer was then precipitated by dropwise addition of 6M HCl accompanied by stirring and pelleted by centrifugation (1,500×g for 5 min). The pellet was then washed 3 times with 50 mL of 25 mM HCl, followed by a third wash in ultrapure water and subsequent lyophilization. Lyophilized SMA was later re-suspended at 10% wt/vol in 25 mM Tris-HCl, and the pH of the solution adjusted to 8 with 1M NaOH. Peptide hydrophobic moment and electropotential was calculated using the 3D-HM calculator (S. Reißer, E. Strandberg, T. Steinbrecher, A. S. Ulrich. Biophysical Journal 2014, 106(11), 2385-2394). Sequences corresponding to NSP or NSPᵣ were calculated with the C-terminus specified as (COO⁻) and N-terminus specified as (NH3⁺). UV absorbance of solubilized peptides was measured by NanoDrop™ spectrophotometer.

### Example 1.0

### The Characteristics of Different Amphipathic Peptide Variants Tested in Experiment #1

The biophysical, structural and experimental characteristics of different amphipathic peptide variants were tested to select the best candidates for use as the scaffold peptides. The amino acid sequences of each of the tested amphipathic peptide variants are listed in Table 5. The ideal scaffold peptide candidates have high peptide solubility and have binding affinities that allow for strong binding to a target membrane protein. In some embodiments, preferred amphipathic scaffold peptide candidates will remain stably bound to the target membrane protein upon prolonged incubation at elevated temperatures.

Peptide solubility can be determined by calculating the hydrophobic moment of the peptide and the electrostatic potential on the peptide surface using the 3D Hydrophobic Moment Vector Calculator. A low hydrophobic moment, high overall electropotential and high solvent accessible area are factors which suggest that the peptide has high solubility in water. Table 6 summarizes the structural characteristics of certain amphipathic peptide variants. Table 7 summarizes the biophysical characteristics of certain amphipathic peptide variants.

TABLE 5
Amino Acid Sequences of Scaffold Peptides Tested in Experiment
#1

| Name of | |
|---|---|
| Sequence | Sequence |
| NSPr | FAEKFKEAVKDYFAKFWD P AAEKLKEAVKDYFAKLWD |
| | (SEQ ID NO: 2) |
| 2F-2F | FAEKLKEAVKDYFAKLWD P FAEKLKEAVKDYFAKLWD |
| | (SEQ ID NO: 3) |
| 4F-4F | FAEKFKEAVKDYFAKFWD P FAEKFKEAVKDYFAKFWD |
| | (SEQ ID NO: 4) |
| NSPr-G | FAEKFKEAVKDYFAKFWD G AAEKLKEAVKDYFAKLWD |
| | (SEQ ID NO: 5) |
| NSPr-3P | FAEKFKEAVKPYFAKFWD P AAEKLKEAVKPYFAKLWD |
| | (SEQ ID NO: 6) |
| NSPr-C | C FAEKFKEAVKDYFAKFWD P AAEKLKEAVKDYFAKLWD |
| | (SEQ ID NO: 7) |
| NSPr-36 | FAEKFKEAVKDYFAKFWD P AAEKLKEAVKDYFAKLW |
| | (SEQ ID NO: 8) |
| NSPr-6xHis | HHHHHH FAEKFKEAVKDYFAKFWD P AAEKLKEAVKDYFAK |
| | LWD |
| | (SEQ ID NO: 9) |
| Ac-NSP-NH₂ | Ac-DWLKAFYDKVAEKLKEAA P DWFKAFYDKVAEKFKEAF- |
| | NH₂ |
| | (SEQ ID NO: 10) |
| NSP | DWLKAFYDKVAEKLKEAA P DWFKAFYDKVAEKFKEAF |
| | (SEQ ID NO: 11) |
| NSPr-FAM | Fam-FAEKFKEAVKDYFAKFWD P AAEKLKEAVKDYFAKLWD |
| | (SEQ ID NO: 12) |
| NSPr-biotin | Bio-FAEKFKEAVKDYFAKFWD P AAEKLKEAVKDYFAKLWD |
| | (SEQ ID NO: 13) |
| NSPr-pyrene | FAEKFKEAVKDYFAKFWD P AAEKLKEAVKDYFAKLWD- |
| | pyrenebutyric acid |
| | (SEQ ID NO: 14) |

TABLE 6
Structural Characteristics of Peptides Tested in Experiment #1

| | | | | |
|---|---|---|---|---|
| | Peptide | | # Coil | # Helix |
| Peptide | Configuration | SEQ ID NO: | Residues | Residues |

Coil/Helix Prediction per Residue

| | | | | |
|---|---|---|---|---|
| NSPr | 4F-Pro-1F | SEQ ID NO: 2 | 6 | 31 |

CCHHHHHHHHHHHHHHCCHHHHHHHHHHHHHHHHHCC

| | | | | |
|---|---|---|---|---|
| 2F-2F | 2F-Pro-2F | SEQ ID NO: 3 | 3 | 34 |

CHHHHHHHHHHHHHHHHHHHHHHHHHHHHHHHHHHCC

| | | | | |
|---|---|---|---|---|
| 4F-4F | 4F-Pro-4F | SEQ ID NO: 4 | 4 | 33 |

CHHHHHHHHHHHHHHHHCHHHHHHHHHHHHHHHHHCC

| | | | | |
|---|---|---|---|---|
| NSPr-G | 4F-Gly-4F | SEQ ID NO: 5 | 6 | 31 |

CCHHHHHHHHHHHHHHHCCHHHHHHHHHHHHHHHHCC

| | | | | |
|---|---|---|---|---|
| NSPr-3P | 2F-Pro-2F-Pro-Pro-1F | SEQ ID NO: 6 | 6 | 31 |

CCHHHHHHHHHHHHHHCCHHHHHHHHHHHHHHHHHCC

| | | | | |
|---|---|---|---|---|
| NSPr-C | 4F-Pro-1F-C | SEQ ID NO: 7 | 5 | 33 |

CHHHHHHHHHHHHHHHHCCHHHHHHHHHHHHHHHHHCC

| | | | | |
|---|---|---|---|---|
| NSPr-36 | 4F-Pro-1F (36 a.a.) | SEQ ID NO: 8 | 5 | 31 |

CCHHHHHHHHHHHHHHCCHHHHHHHHHHHHHHHHHC

| | | | | |
|---|---|---|---|---|
| NSPr-6xHis | 6xH-4F-Pro-1F | SEQ ID NO: 9 | 7 | 36 |

CCCHHHHHHHHHHHHHHHHHHHCCHHHHHHHHHHHHHHHHHCC

| | | | | |
|---|---|---|---|---|
| Ac-NSP-NH₂ | Ace-1F-Pro-4F-Amid | SEQ ID NO: 10 | 5 | 32 |

CHHHHHHHHHHHHHHHHCCHHHHHHHHHHHHHHHHCC

| | | | | |
|---|---|---|---|---|
| NSP | 1F-Pro-4F | SEQ ID NO: 11 | 5 | 32 |

CHHHHHHHHHHHHHHHHCCHHHHHHHHHHHHHHHHCC

| | | | |
|---|---|---|---|
| NSPr-FAM | Fam-4F-Pro-1F | SEQ ID NO: 12 | nd |
| NSPr-biotin | Bio-4F-Pro-1F | SEQ ID NO: 13 | nd |
| NSPr-pyrene | 4F-Pro-1F-Pyr | SEQ ID NO: 14 | nd |

TABLE 7
Biophysical Characteristics of Peptides Tested in Experiment #1

| | | Absolute | Average Absolute | Solvent | | |
|---|---|---|---|---|---|---|
| | | Hydrophobic | Electropotential on | accessible | Salt | |
| Peptide | Peptide | Moment (A*kT/e) | Surface (kT/e) | area (A\hat{ }2) | bridges | Charge |
| NSPr | 4F-Pro-1F | 5.792 | 5.234 | 3462.435 | 17 | 0 |
| 2F-2F | 2F-Pro-2F | 5.718 | 4.680 | 3170.398 | 18 | 0 |
| 4F-4F | 4F-Pro-4F | 5.455 | 4.755 | 3358.815 | 19 | 0 |
| NSPr-G | 4F-Gly-1F | 5.893 | 4.945 | 3242.681 | 18 | 0 |
| NSPr-3P | 2F-Pro-2F-Pro-Pro-1F | 5.079 | 4.811 | 3478.264 | 12 | 2 |
| NSPr-C | 4F-Pro-1F-C | 5.148 | 4.885 | 3510.825 | 16 | 0 |
| NSPr-36 | 4F-Pro-1F (36 a.a.) | 4.771 | 4.771 | 3289.665 | 20 | 1 |
| NSPr-6xHis | 6xH-4F-Pro-1F | 5.853 | 4.865 | 3741.474 | 22 | 1 |
| Ac-NSP-NH₂ | Ace-1F-Pro-4F-Amid | 5.532 | 5.026 | 3009.359 | 15 | −1 |
| NSP | 1F-Pro-4F | 6.130 | 5.066 | 3359.007 | 15 | 0 |
| NSPr-FAM | Fam-4F-Pro-1F | nd | nd | nd | nd | −1 |
| NSPr-biotin | Bio-4F-Pro-1F | nd | nd | nd | nd | −1 |
| NSPr-pyrene | 4F-Pro-1F-Pyr | nd | nd | nd | nd | −1 |

Table 8 summarizes the experimental characteristics of certain amphipathic peptide variants. The solubility, reconstitution efficiency and competition efficiency of each of the listed amphipathic peptide variants were determined. The solubility of the peptides was determined by measuring the turbidity of each of the peptide suspensions at 550 nm. The numbers 1 to 5 were used denote the degree of solubility, where 1 means poor solubility and 5 means high solubility. The reconstitution efficiency of the peptides was determined by assessing by an on-gel reconstitution method the ability of each of the peptide variants to reconstitute the membrane proteins MsbA and FhuA. The numbers 1 to 5 were used to denote the efficiency of reconstitution, where 1 means poor reconstitution and 5 means efficient reconstitution. The competition efficiency of the peptides was determined by adding equimolar amounts of NSPᵣ-FAM and variants during reconstitution of membrane proteins by the on-gel reconstitution method. The numbers 1 to 100 were used to denote the level of competition, where 0 means no competition and 100 means full competition.

TABLE 8
Experimental Characteristics of Peptides Tested in Experiment #1

| | | | | Reconstitution | Competition |
|---|---|---|---|---|---|
| | | | | Efficiency | peptide |
| Peptide | Peptide | SEQ ID NO | Solubility | (/5) | (% NSPr-FAM) |
| NSPr | 4F-Pro-1F | SEQ ID NO: 2 | 5 | 5 | 100 |
| 2F-2F | 2F-Pro-2F | SEQ ID NO: 3 | 3 | 4 | 70 |
| 4F-4F | 4F-Pro-4F | SEQ ID NO: 4 | 3 | 3 | 60 |
| NSPr-G | 4F-Gly-1F | SEQ ID NO: 5 | 5 | 2 | 10 |
| NSPr-3P | 2F-Pro-2F-Pro-Pro-1F | SEQ ID NO: 6 | 5 | 1 | 0 |
| NSPr-C | 4F-Pro-1F-C | SEQ ID NO: 7 | 5 | 5 | 100 |
| NSPr-36 | 4F-Pro-1F (36 a.a.) | SEQ ID NO: 8 | 3 | 4 | 60 |
| NSPr-6xHis | 6xH-4F-Pro-1F | SEQ ID NO: 9 | 3 | 4 | 40 |
| Ac-NSP-NH₂ | Ace-1F-Pro-4F-Amid | SEQ ID NO: 10 | 1 | 2 | 10 |
| NSP | 1F-Pro-4F | SEQ ID NO: 11 | 3 | 3 | 40 |
| NSPr-FAM | Fam-4F-Pro-1F | SEQ ID NO: 12 | 5 | 5 | X |
| NSPr-biotin | Bio-4F-Pro-1F | SEQ ID NO: 13 | 5 | 5 | n.d. |
| NSPr-pyrene | 4F-Pro-1F-Pyr | SEQ ID NO: 14 | 5 | 4 | n.d. |

### Example 2.0

### The Characteristics of Different Amphipathic Peptide Variants Tested in Experiment #2

The biophysical and experimental characteristics of a different set of amphipathic peptide variants were tested to select the best candidates for use as the scaffold peptides. The amino acid sequences of each of the amphipathic peptide variants tested in Experiment #2 are listed in Table 9.

TABLE 9
Amino Acid Sequences of Scaffold Peptides Tested in Experiment
#2

| Name of | |
|---|---|
| Sequence | Sequence |
| NSPr | FAEKFKEAVKDYFAKFWD P AAEKLKEAVKDYFAKLWD |
| | (SEQ ID NO: 2) |
| 3F-1F | FAEKFKEAVKDYFAKLWD P AAEKLKEAVKDYFAKLWD |
| | (SEQ ID NO: 15) |
| 4F-2F | FAEKFKEAVKDYFAKFWD P AAEKLKEAVKDYFAKFWD |
| | (SEQ ID NO: 16) |
| 4F-3F | FAEKFKEAVKDYFAKFWD P AAEKFKEAVKDYFAKLFD |
| | (SEQ ID NO: 17) |
| 4F-4F | FAEKFKEAVKDYFAKFWD P FAEKFKEAVKDYFAKFWD |
| | (SEQ ID NO: 4) |
| 2F-2F | FAEKLKEAVKDYFAKLWD P FAEKLKEAVKDYFAKLWD |
| | (SEQ ID NO: 5) |
| 2F-1F | FAEKLKEAVKDYLAKFWD P AAEKLKEAVKDYFAKLWD |
| | (SEQ ID NO: 18) |
| Neg Switch | FADKFKDAVKEYFAKFWE P AADKLKDAVKEYFAKLWE |
| | (SEQ ID NO: 19) |
| Pos Switch | FAERFREAVRDYFARFWD P AAERLREAVRDYFARLWD |
| | (SEQ ID NO: 20) |
| Phe/Trp | WAEKFKEAVKDYWAKFWD P AAEKLKEAVKDYWAKLWD |
| Switch | (SEQ ID NO: 21) |
| Phe/Tyr | YAEKFKEAVKDYFAKFYD P AAEKLKEAVKDYFAKLWD |
| Switch | (SEQ ID NO: 22) |
| Ala/Val | FVEKFKEVAKDYFVKFWD P VVEKLKEVAKDYFVKLWD |
| Switch | (SEQ ID NO: 23) |
| Tyr/Gln | FAEKFKEAVKDQFAKFWD P AAEKLKEAVKDQFAKLWD |
| Switch | (SEQ ID NO: 24) |
| 1 SB | FAEAFKEAVKDYFAKFWD P AAEKLKEAVKDYFAKLWD |
| Disruption | (SEQ ID NO: 25) |
| 2 SB | FAEAFKEAVKDYFAAFWD P AAEKLKEAVKDYFAKLWD |
| Disruption | (SEQ ID NO: 26) |
| 3 SB | FAEAFKEAVKDYFAAFWD P AAEKLKEAVADYFAKLWD |
| Disruption #1 | (SEQ ID NO: 27) |
| 3 SB | FAAKFKEAVKDYFAKFWD P AAEKLAEAVADYFAALWD |
| Disruption #2 | (SEQ ID NO: 28) |
| Pro/Gly | FAEKFKEAVKDYFAKFWD G AAEKLKEAVKDYFAKLWD |
| Switch | (SEQ ID NO: 29) |
| NSPr-3P | FAEKFKEAVKPYFAKFWD P AAEKLKEAVKPYFAKLWD |
| | (SEQ ID NO: 6) |
| NSP | DWLKAFYDKVAEKLKEAA P DWFKAFYDKVAEKFKEAF |
| | (SEQ ID NO: 11) |
| Ac-NSP-NH₂ | Ac-DWLKAFYDKVAEKLKEAA P DWFKAFYDKVAEKFKEAF- |
| | NH₂ |
| | (SEQ ID NO: 10) |
| NSPr-C | C FAEKFKEAVKDYFAKFWD P AAEKLKEAVKDYFAKLWD |
| | (SEQ ID NO: 7) |
| NSPr-36 | FAEKFKEAVKDYFAKFWD P AAEKLKEAVKDYFAKLW |
| | (SEQ ID NO: 8) |
| NSPr-6xHis | HHHHHH FAEKFKEAVKDYFAKFWD P AAEKLKEAVKDYFAK |
| | LWD |
| | (SEQ ID NO: 9) |
| NSPr-FAM | Fam-FAEKFKEAVKDYFAKFWD P AAEKLKEAVKDYFAKLWD |
| | (SEQ ID NO: 12) |
| NSPr-Biotin | Bio-FAEKFKEAVKDYFAKFWD P AAEKLKEAVKDYFAKLWD |
| | (SEQ ID NO: 13) |
| NSPr-Pyrene | FAEKFKEAVKDYFAKFWD P AAEKLKEAVKDYFAKLWD- |
| | pyrenebutyric acid |
| | (SEQ ID NO: 14) |

Table 10 summarizes the biophysical, structural and experimental characteristics of the tested NSP peptide variants. Specifically, the reconstitution efficiency, solubility, competition efficiency, hydrophobic moment and surface electropotential of each of the listed amphipathic peptide variants were determined using the same methods as described above in Example 1.0. The number of inter-helix salt bridges formed between the AH₁ and AH₂ regions for each of the tested amphipathic peptide variants was also determined.

TABLE 10
Biophysical, Structural and Experimental Characteristics of Peptides Tested in Experiment #2

| | Reconstitution | | Competition | # of inter- | Hydrophobic | Surface |
|---|---|---|---|---|---|---|
| | Efficiency | Solubility | NSPr-FAM | Helix Salt | Moment | Electropotential |
| Peptide | (/5) | (/5) | (±10%) | Bridges | (A*kT/e) | (kT/e) |
| 4F-1F (NSPr) | 5 | 5 | 100 | 60 | 5.792 | 5.234 |
| SEQ ID NO: 2 | | | | | | |
| 3F-1F | 5 | 5 | 100 | 52 | 5.498 | 4.896 |
| SEQ ID NO: 15 | | | | | | |
| 4F-2F | 5 | 4 | 90 | 64 | 6.345 | 4.799 |
| SEQ ID NO: 16 | | | | | | |
| 4F-3F | 4 | 4 | 70 | 60 | 5.231 | 4.899 |
| SEQ ID NO: 17 | | | | | | |
| 4F-4F | 4 | 4 | 60 | 54 | 5.455 | 4.755 |
| SEQ ID NO: 4 | | | | | | |
| 2F-2F | 4 | 4 | 70 | 50 | 5.718 | 4.680 |
| SEQ ID NO: 5 | | | | | | |
| 2F-1F | 3 | 5 | 60 | 64 | 5.488 | 4.884 |
| SEQ ID NO: 18 | | | | | | |
| Neg Switch | 4 | 4 | 60 | 56 | 5.730 | 4.889 |
| SEQ ID NO: 19 | | | | | | |
| Pos Switch | 4 | 5 | 90 | 52 | 5.197 | 4.861 |
| SEQ ID NO: 20 | | | | | | |
| Phe/Trp Switch | 3 | 4 | 70 | 54 | 7.271 | 4.821 |
| SEQ ID NO: 21 | | | | | | |
| Phe/Tyr Switch | 3 | 4 | 70 | 62 | 8.094 | 4.788 |
| SEQ ID NO: 22 | | | | | | |
| Ala/Val Switch | 4 | 5 | 80 | 60 | 7.475 | 4.686 |
| SEQ ID NO: 23 | | | | | | |
| Tyr/Gln Switch | 4 | 4 | 80 | 70 | 7.857 | 4.826 |
| SEQ ID NO: 24 | | | | | | |
| 1 SB Disruption | 4 | 5 | 80 | 54 | 4.948 | 4.526 |
| SEQ ID NO: 25 | | | | | | |
| 2 SB Disruption | 5 | 5 | 70 | 48 | 5.171 | 4.534 |
| SEQ ID NO: 26 | | | | | | |
| 3 SB Disruption H1 | 2 | 4 | 30 | 40 | 8.130 | 4.978 |
| SEQ ID NO: 27 | | | | | | |
| 3 SB Disruption H2 | 2 | 5 | 40 | 64 | 6.267 | 5.250 |
| SEQ ID NO: 28 | | | | | | |
| Pro/Gly Switch | 5 | 4 | 80 | 60 | 5.893 | 4.945 |
| SEQ ID NO: 29 | | | | | | |
| NSPr-3P | 1 | 5 | 0 | 62 | 5.079 | 4.811 |
| SEQ ID NO: 6 | | | | | | |
| NSPr-Inv(NSP) | 3 | 3 | 40 | 64 | 6.130 | 5.066 |
| SEQ ID NO: 11 | | | | | | |
| Ac-NSPr-Inve-NH₂ | 2 | 1 | 10 | n.d. | 5.532 | 5.026 |
| (Ac-NSP-NH₂) | | | | | | |
| SEQ ID NO: 10 | | | | | | |
| NSPr-C | 5 | 5 | 100 | 58 | 5.148 | 4.885 |
| SEQ ID NO: 7 | | | | | | |
| NSPr-36 | 5 | 3 | 60 | 64 | 4.771 | 4.771 |
| SEQ ID NO: 8 | | | | | | |
| NSPr-6xHis | 4 | 3 | 40 | 96 | 5.853 | 4.865 |
| SEQ ID NO: 9 | | | | | | |
| NSPr-FAM | 5 | 5 | n.d. | n.d. | n.d. | n.d. |
| SEQ ID NO: 12 | | | | | | |
| NSPr-Biotin | 5 | 5 | n.d. | n.d. | n.d. | n.d. |
| SEQ ID NO: 13 | | | | | | |
| NSPr-Pyrene | 4 | 5 | n.d. | n.d. | n.d. | n.d. |
| SEQ ID NO: 14 | | | | | | |

Table 11 summarizes the Gibbs free energy (i.e., an indicator of the stability of the three dimensional structure of a peptide) and the hydrophobic moment for the total scaffold peptide and for each of the AH₁ and AH₂ regions for each of the tested amphipathic scaffold peptides. Both data sets featured in Table 11 were generated using Membrane Protein Explorer (MPEx), a computational tool that models peptides based on the hydrophobicity/hydrophilicity of their individual constitutive amino acids. MPEx incorporates both calculated and experimentally derived amino acid hydropathy plots to provide an overall model of peptide stability and hydrophobicity.

TABLE 11
Biophysical, Structural and Experimental Characteristics
of Peptides Tested in Experiment #2

| | | | | Hydrophobic | | |
|---|---|---|---|---|---|---|
| | ΔG (kcal/mol) | | | Moment (kcal/mol) | | |
| Peptide | Total | Helix 1 | Helix 2 | Total | Helix 1 | Helix 2 |
| 4F-1F (NSPr) | 37.55 | 17.14 | 20.27 | 24.44 | 21.2 | 19.59 |
| 3F-1F | 38.01 | 17.6 | 20.27 | 24.2 | 20.74 | 19.59 |
| 4F-2F | 37.93 | 17.14 | 20.65 | 24.75 | 21.2 | 19.57 |
| 4F-3F | 37.47 | 17.14 | 20.19 | 25.17 | 21.2 | 19.96 |
| 4F-4F | 34.42 | 17.14 | 17.14 | 27.32 | 21.2 | 21.2 |
| 2F-2F | 36.26 | 18.06 | 18.06 | 26.2 | 20.33 | 20.33 |
| 2F-1F | 38.47 | 18.06 | 20.27 | 23.88 | 20.51 | 19.59 |
| Neg Switch | 37.55 | 17.14 | 20.27 | 24.44 | 21.2 | 19.58 |
| Pos Switch | 29.63 | 13.18 | 16.31 | 23.18 | 20.22 | 18.62 |
| Phe/Trp Switch | 36.41 | 16.38 | 19.89 | 24.56 | 21.58 | 19.85 |
| Phe/Tyr Switch | 39.93 | 19.52 | 20.27 | 23.89 | 20.89 | 19.59 |
| Ala/Val Switch | 32.75 | 15.22 | 17.39 | 24.16 | 20.27 | 18.94 |
| Tyr/Gln Switch | 40.51 | 18.62 | 21.75 | 23.19 | 20.21 | 18.74 |
| 1 SB Disruption | 35.25 | 14.84 | 20.27 | 22.15 | 19.91 | 19.59 |
| 2 SB Disruption | 32.95 | 12.54 | 20.27 | 20.05 | 19.6 | 19.59 |
| 3 SB Disruption H1 | 30.65 | 12.54 | 17.97 | 17.83 | 19.6 | 19.05 |
| 3 SB Disruption H2 | 27.52 | 14.01 | 13.37 | 22.95 | 19.18 | 19.05 |
| Pro/Gly Switch | 38.56 | 17.14 | 20.27 | 24.92 | 21.2 | 19.59 |
| NSPr-3P | 30.55 | 13.64 | 16.77 | 20.81 | 18.38 | 16.53 |
| NSPr-Inv | 37.55 | 20.27 | 17.14 | 24.44 | 19.59 | 21.2 |
| Ac-NSPr-Inve-NH₂ | 39.55 | 22.27 | 19.14 | 24.44 | 19.59 | 21.2 |
| NSPr-C | 37.53 | 13.48 | 20.27 | 24.43 | 17.66 | 19.59 |
| NSPr-36 | 33.91 | 17.14 | 16.77 | 23.01 | 21.2 | 16.12 |
| NSPr-6xHis | 51.53 | 29.69 | 20.27 | 23.8 | 17.61 | 19.59 |
| NSPr-FAM | n.d. | n.d. | n.d. | n.d. | n.d. | n.d. |
| NSPr-Biotin | n.d. | n.d. | n.d. | n.d. | n.d. | n.d. |
| NSPr-Pyrene | n.d. | n.d. | n.d. | n.d. | n.d. | n.d. |

The foregoing results demonstrate that substitution of the aromatic amino acid phenylalanine with other aromatic amino acids such as tryptophan or tyrosine still allows the amphipathic scaffold peptide to retain a high degree of solubility and to reconstitute membrane proteins. Substituting a positive or a negative residue that participates in salt bridge formation with a different positive or negative residue still allows the amphipathic scaffold peptide to retain a high degree of solubility and to reconstitute membrane proteins. Varying the number of aromatic amino acids in the sequence or their ratio including in the ratios in AH₁:AH₂ of 4:1, 3:1, 4:2, 4:3, 4:4, 2:2, or 2:1 still allows the amphipathic scaffold peptide to retain a high degree of solubility and to reconstitute membrane proteins. Interchanging a small hydrophobic amino acid such as alanine for another small hydrophobic amino acid such as valine still allows the amphipathic scaffold peptide to retain a high degree of solubility and to reconstitute membrane proteins. Interchanging a polar amino acid such as tyrosine for another polar amino acid such as glutamine still allows the amphipathic scaffold peptide to retain a high degree of solubility and to reconstitute membrane proteins. Disrupting one or two intra-helical salt bridges within the amphipathic scaffold peptide still allows the amphipathic scaffold peptide to retain a high degree of solubility and to reconstitute membrane proteins. Substituting the small amino acid at the linker position for another small amino acid at the linker position, e.g. substituting glycine for proline, still allows the amphipathic scaffold peptide to retain a high degree of solubility and to reconstitute membrane proteins. Adding a label or affinity tag to the amphipathic scaffold peptide still allows the amphipathic scaffold peptide to retain a high degree of solubility and to reconstitute membrane proteins. Adding a cysteine residue to the amphipathic scaffold peptide still allows the amphipathic scaffold peptide to retain a high degree of solubility and to reconstitute membrane proteins. Removing the charged amino acid residue at the C-terminus of the amphipathic scaffold peptide still allows the amphipathic scaffold peptide to retain a high degree of solubility and to reconstitute membrane proteins.

In some embodiments, the amphipathic scaffold peptide does not have the amino acid sequence of SEQ ID NO: 10. In some embodiments, the amphipathic scaffold peptide does not have the amino acid sequence of SEQ ID NO: 11.

### Example 3.0

### Solubility Test of NSP and NSPᵣ

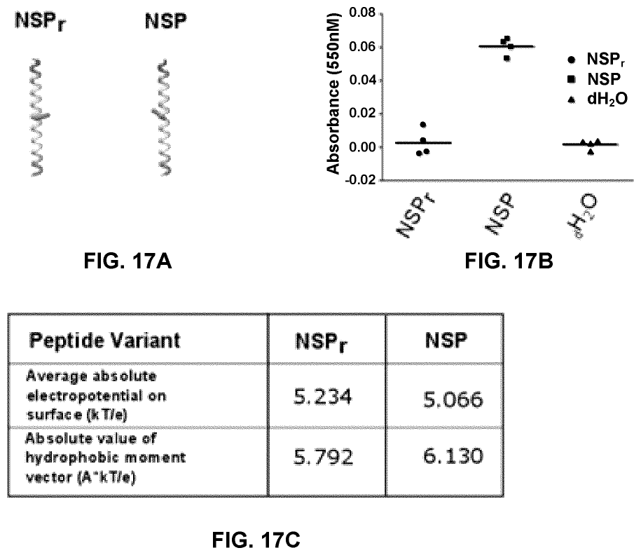

Peptide models of NSP and NSPᵣ are computed by a 3D-hydrophobic moment peptide calculator. The amino acid sequences of NSP and NSPᵣ are SEQ ID NO: 11 and SEQ ID NO: 2 respectively. The direction of hydrophobic moment is indicated by a diagonal line near the centre of each peptide as shown in FIG. 17A. The peptides are oriented with their N to C-terminus from bottom to top. FIG. 17B illustrates the turbidity measurements of peptide suspension of each of NSP and NSPᵣ suspended in distilled water (dH₂O). FIG. 17B shows the absorbance of light at 550 nm for NSP (at 5 mg/ml, squares, centre data) and NSPᵣ (at 25 mg/ml, circles, left side data) as compared to a dH₂O control (triangles, right side data). FIG. 17B shows that NSPᵣ is fully dissolved in water at concentrations up to 25 mg/mL. The table in FIG. 17C lists the calculated electropotential and hydrophobic moment of NSP and NSPᵣ. Peptide hydrophobic moment and electropotential were calculated using a 3D-HM calculator as discussed in S. Reißer, E. Strandberg, T. Steinbrecher, A. S. Ulrich. Biophysical Journal 2014, 106(11), 2385-2394.

### Example 4.0

### “On-Column” Reconstitution of MalFGK₂

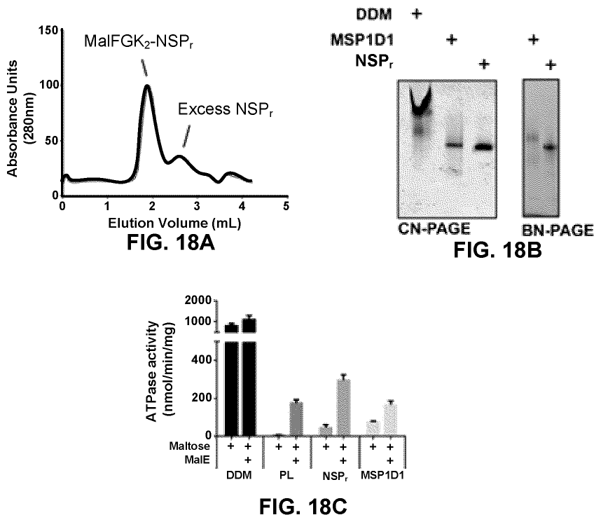

The ability of the scaffold peptide with the amino acid sequence SEQ ID NO: 2 (NSPᵣ) to capture the ABC transporter MalFGK₂ was tested using an “on-column” reconstitution method. The NSPᵣ peptide was mixed with MalFGK₂ in dodecyl maltoside (DDM) as a solubilizing agent and the mixture applied immediately onto a size exclusion column equilibrated in a detergent-free buffer. The results of the size-exclusion chromatography of MalFGK₂ in peptidisc (MalFGK₂-NSR) are shown in FIG. 18A. FIG. 18B illustrates the CN-PAGE (clear native PAGE) and BN-PAGE (blue native PAGE) analysis of MalFGK₂ in detergent micelle (DDM), nanodisc (MSP1D1), and peptidisc. The CN-PAGE and BN-PAGE show that the collected particles (MalFGK₂-NSPᵣ) were soluble and monodisperse. The maltose-dependent ATPase activity of MalFGK₂ reconstituted in each of detergent (DDM), proteoliposomes (PL), peptidiscs, and nanodiscs (MSP1D1) obtained at 30° C. in the presence or absence of MalE were compared. With reference to FIG. 18C, the ATPase activity of MalFGK₂ in peptidisc was similar to that reported in proteoliposomes and in nanodiscs, as reported in H Bao, F Duong. PLoS ONE 2012, 7:e34836. With reference to FIG. 18C, the ATPase activity of MalFGK₂ in peptidisc is in sharp contrast to the high and unregulated ATPase activity observed in detergent micelles.

### Example 5.0

### “In-Gel” Method for Determining Optimal Reconstitution Ratio

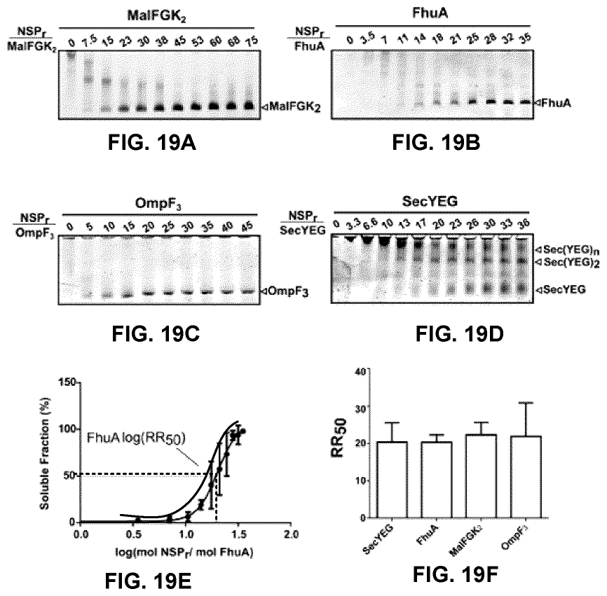

The ‘in-gel’ method was developed to determine optimal reconstitution conditions in a time and cost-effective manner. Small amounts of amphipathic scaffold peptides (0-2.5 μg) were mixed with the target protein (˜1.25 μg) in detergent solution, and the resulting mixture immediately loaded on native gel. Removal of the non-ionic detergent occurs during electrophoresis when the protein-peptide mixture enters the detergent-free part of the gel. The effective NSPᵣ concentrations required to trap four different integral membrane complexes into a peptidisc were estimated for each of MalFGK₂ (FIG. 19A), FhuA (FIG. 19B), the trimeric OmpF porin (FIG. 19C), and the membrane translocon SecYEG (FIG. 19D).

FIG. 19E illustrates the reconstitution efficiency of FhuA as a function of the NSPᵣ concentration. To obtain the data, the protein band FhuA-NSP in FIG. 19B was quantified with Image J and the data plotted as log (mol NSP/mol FhuA). The data were fitted with a Boltzmann sigmoidal function to generate a curve describing the reconstitution efficiency and the half-maximal reconstitution ratio (RR₅₀). The RR₅₀ was determined for other target proteins, as shown in FIG. 19F. With reference to FIGS. 19E and 19F, the tested membrane proteins were generally reconstituted at similar peptide concentrations, with a half-maximal molar ratio (RR₅₀) of 20. This is significantly higher than the measured stoichiometry of about 10 peptides per protein complex suggesting that excess peptide is needed to achieve efficient assembly. This analysis also showed that the SecYEG complex may be trapped as a dimer and higher order oligomeric form in peptidisc (FIG. 19D). It is hypothesized that the dimer and higher order oligomeric forms are possible due to the self-association of this complex in detergent solution. This observation further differentiates peptidiscs from nanodiscs. In nanodiscs, the selective reconstitution of the SecYEG monomer and dimer requires MSP proteins of different lengths.

### Example 6.0

### Direct “On-Beads” Reconstitution

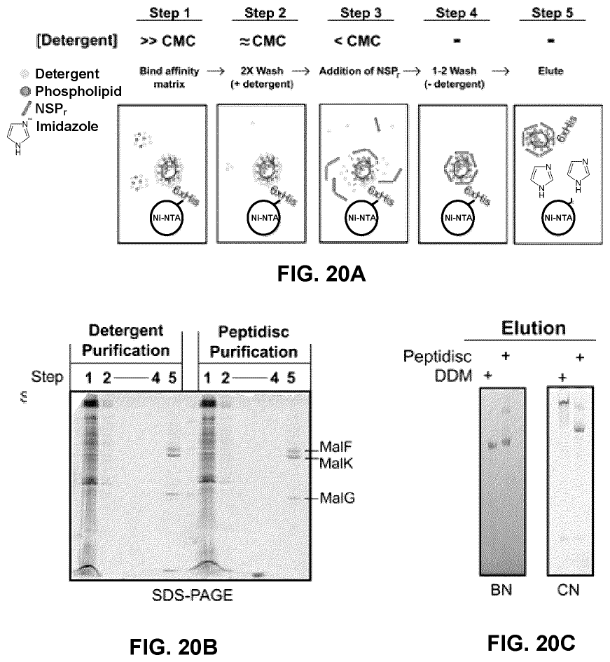

FIG. 20A is a schematic diagram illustrating the steps of reconstituting using the tested on-beads method. Step 1 involves extracting the tagged protein from the membrane with excess of detergent buffer (greater than its critical micellization concentration or “CMC”) and incubating with an affinity resin. Step 2 involves washing the beads twice with the detergent buffer near its CMC. Step 3 involves incubating the beads with buffer containing excess amphipathic scaffold peptide and limited amount of detergent (less than its CMC). Step 4 involves washing the beads in detergent-free buffer to remove unbound amphipathic scaffold peptide and residual detergent. Step 5 involves eluting the protein captured as peptidiscs from the column in detergent-free solution. FIGS. 20B and 20C are, respectively, SDS-PAGE and Native-PAGE analysis of the His-tagged MalFGK₂ complex purified following conventional detergent method and “on-beads” peptidisc detergent-free method. Analysis of the eluted complex by BN-PAGE and CN-PAGE showed that MalFGK₂ is readily incorporated into peptidiscs, with purity and yield as good as with conventional detergent-based chromatography.

### Example 7.0

### Thermostability of the BRC Complex in Peptidiscs

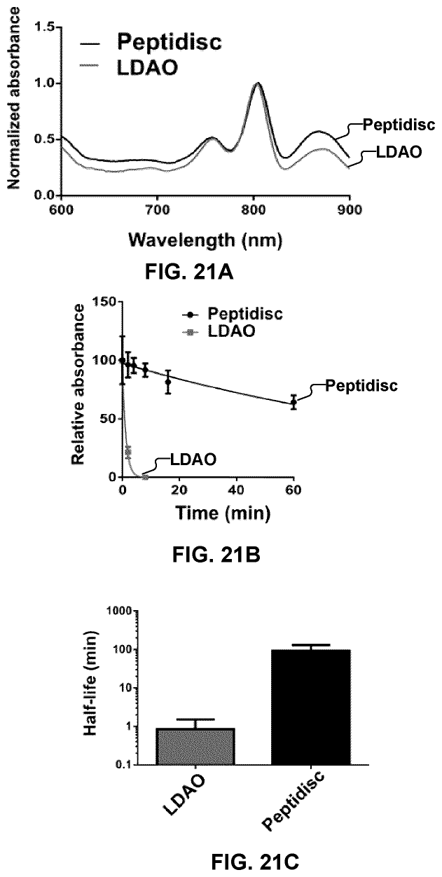

The photosynthetic bacterial reaction center (BRC) from Rhodobacter sphaeroides was reconstituted into peptidiscs using NSPᵣ. FIG. 21A illustrates the absorbance scans of the BRC (1 μM) in detergent solution (0.03% LDAO (lauryldimethylamine oxide)) and in peptidisc. Scans were normalized to the value measured at 803 nm (the absorbance peak of the accessory bacteriochlorophylls). The spectral properties of the BRC complex were similar in both environments. However, the BRC in peptidisc resisted denaturation at 65° C. for 1 hour, while it was fully denatured in less than 4 minutes in LDAO. As shown in FIG. 21B, a decrease in absorbance of the BRC at 803 nm was observed after incubation at 65° C. for 1 hour. FIG. 21C is the calculated half-life of the BRC in peptidisc and LDAO at 65° C.

### Example 8.0

### Quantification of NSPᵣ in Peptidiscs

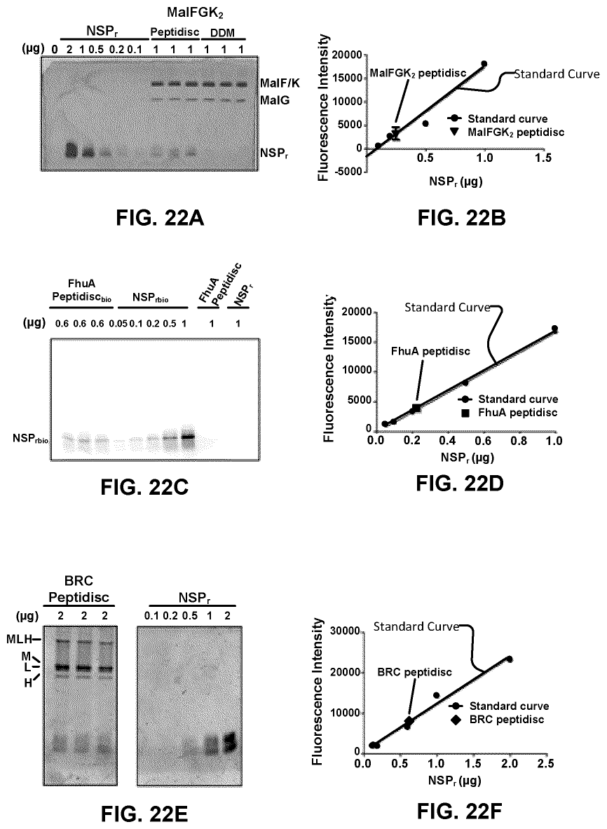

The approximate peptide contents of MalFGK₂ in peptidisc or DDM was determined using 15% SDS-PAGE as shown in FIG. 22A. NSPᵣ runs at the bottom of the PAGE gel and can be visualized with Coomassie blue staining. Dye fluorescence was measured on a LICOR Odyssey scanner and quantified by Image J. FIG. 22B is a standard curve derived from NSPᵣ titration measurement (circles), and average intensity of NSPᵣ fluorescence from MalFGK₂ peptidisc (triangle). FIG. 22C is a western blot of FhuA-peptidisc reconstituted using NSPᵣbio (biotinylated NSPᵣ), and visualized by incubation with Streptavidin-Alexa 680. Fluorescence of the Alexa 680 dye was measured on a LICOR Odyssey scanner (700 nm, excitation 680 nm) and quantified in Image J. FIG. 22D is a standard curve derived from NSPᵣ titration measurement (circles), and average intensity of NSPᵣ fluorescence from FhuA peptidisc (square).

FIG. 22E illustrates a 15% SDS-PAGE analysis of BRC in peptidisc. The MLH subunits of BRC partially resist denaturation by SDS, resulting in a higher molecular weight band located above the single subunits. Each gel was repeated in triplicate with independent standard curves to calculate the values reported in Table 12. The formula for the calculated molecular weight is: MW_{peptidisc}=MW₍ₚᵣₒₜₑᵢₙ₎+n(MW_{NSPr})+m(MW_{Lipid}); where n is the measured NSPᵣ stoichiometry, m is the measured lipid stoichiometry, MW_{Lipid}=0.8 kDa, MW_{NSPr}=4.5 kDa, and MWₚᵣₒₜₑᵢₙ=173 kDa, 80 kDa, and 94 kDa for MalFGK₂, FhuA, and BRC, respectively. FIG. 22F is a standard curve derived from NSPᵣ titration measurement (circles), and average intensity of NSPᵣ fluorescence from BRC peptidisc (diamond).

TABLE 12
Calculated and Observed Molecular Weight
and Scaffold Stoichiometry of Peptidiscs

| | Molecular | Measured | Measured | Calcu- |
|---|---|---|---|---|
| | weight | NSPᵣ | lipid | lated |
| | measured by | stoichi- | stoichi- | molecular |
| | ESI-MS | ometry | ometry | weight* |
| Peptidisc | (kDa) | (NSPᵣ/disc) | (Lipid/Disc) | (kDa) |
| MalFGK₂-NSPᵣ | 247 ± 24 | 10 (±2): 1 | 41 (±10): 1 | 251 ± 12 |
| BRC-NSPᵣ | 138 ± 18 | 9 (±1): 1 | 4 (±1): 1 | 138 ± 5 |
| FhuA-NSPᵣ | 137 ± 18 | 10 (±2): 1 | 4 (±1): 1 | 131 ± 9 |

### Example 9.0

### Quantification of Phospholipids Trapped in Peptidiscs

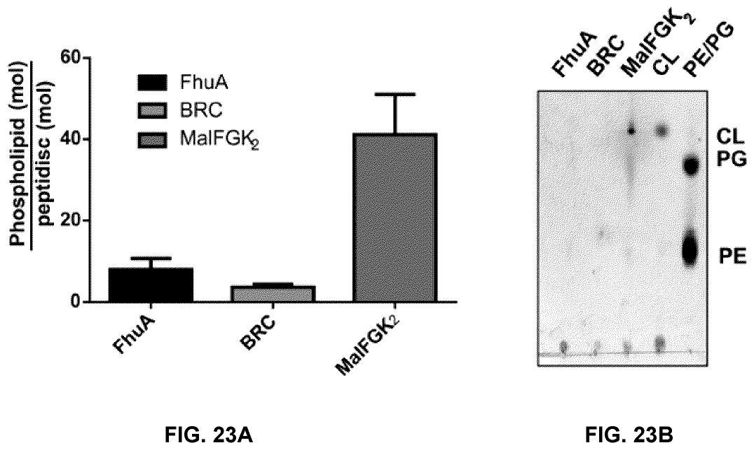

With reference to FIG. 23A, the number of phospholipids per peptidisc was determined in each of FhuA-peptidisc, BRC-peptidisc and MalFGK₂-peptidisc made using NSPᵣ. Phospholipid content was determined by Malachite green assay after acid digestion of lipid extracts. FIG. 23B shows the results from a thin layer chromatography (TLC) analysis of lipid extracts obtained from 10 μg MalFGK₂ peptidisc, 10 μg FhuA peptidisc and 20 μg BRC peptidisc, as well as pure lipid standards Cardiolipin (CL), 1,2-dioleoyl-sn-glycero-3-phosphoglycerol (PG), and 1,2-dioleoyl-sn-glycero-3-phosphoethanolamine (PE). FIG. 23B illustrates that the lipids identified in the TLC analysis were predominantly negative phospholipids, cardiolipin and phosphatidylglycerol. As can be seen in particular based on the results for the MalFGK₂-peptidisc, amphipathic scaffold peptides according to some embodiments can be used to stabilize nanoparticles containing a high level of endogenous and exogenous phospholipids. In some embodiments, stabilization of nanoparticles containing or formed from lipids may be useful, for example to stabilize lipid particles containing various different payloads such as hydrophobic drugs for in vivo delivery.

### Example 10.0

### Stability of MalFGK₂ Peptidisc

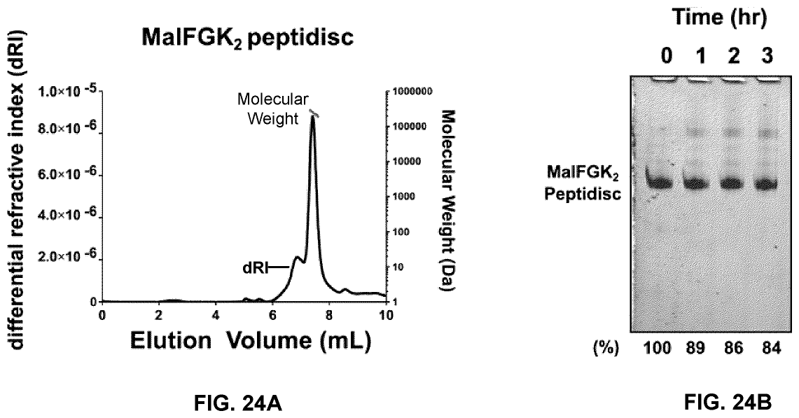

FIG. 24A shows the results from a multi-angle light scattering analysis of MalFGK₂ reconstituted in peptidisc. MalFGK₂-NSPᵣ (100 μg) was left for 3 days at 4° C. before analysis by SEC-MALS. The protein sample was injected and protein concentration was tracked through differential refractive interferometry (dRI). The molecular weight was calculated for the fractions corresponding to the MalFGK₂-NSP peak (larger peak, labelled Molecular Weight. Structural stability of MalFGK₂-NSPᵣ was also determined by BN-PAGE analysis. With reference to FIG. 24B, the structural integrity of the peptidisc remained stable at elevated temperatures, with about 80% of MalFGK₂ peptidisc being intact after incubation for 3 hours at 30° C.

### Example 11.0

### Comparison of Binding Activity of FhuA in Nanodiscs and Peptidiscs

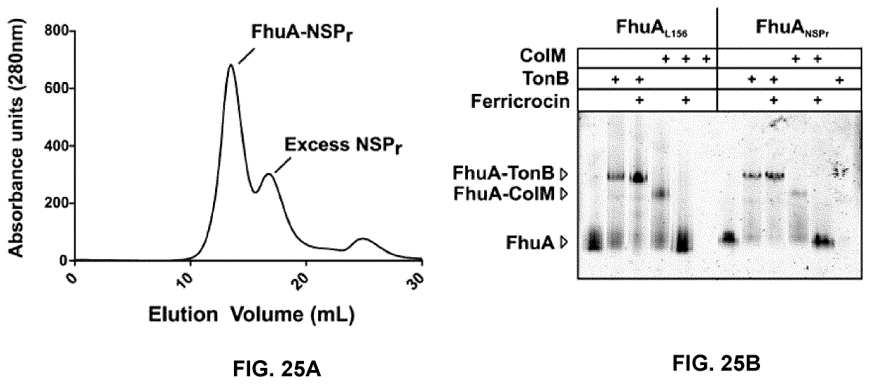

FIG. 25A shows a typical size exclusion chromatography (SEC) profile of FhuA reconstituted in peptidisc (FhuA-NSPᵣ) using an “on-column” reconstitution protocol. The FhuA receptor is a β-barrel membrane protein. The FhuA transporter reconstituted in nanodiscs (FhuA-MSP_{L156}) or peptidiscs (FhuA-NSPᵣ) was incubated with the C-terminal TonB₂₃₋₃₂₉ fragment (2 μg) or with colicin M (5 μg), with or without ferricrocin. Samples were then analysed by CN-PAGE and Coomassie-blue staining of the gel as shown in FIG. 25B. The FIG. 25B binding analysis on CN-PAGE showed that FhuA in peptidisc is functional for both TonB and colicin M. The binding of TonB and colicin M is modulated by the ligand ferricrocin, as previously reported in vivo and in vitro. It is therefore believed that peptidisc is suitable for the functional reconstitution of both α-helical and β-barrel membrane proteins.

### Example 12.0

### Capture of SecYEG Monomer and Dimer in Peptidisc and Nanodisc

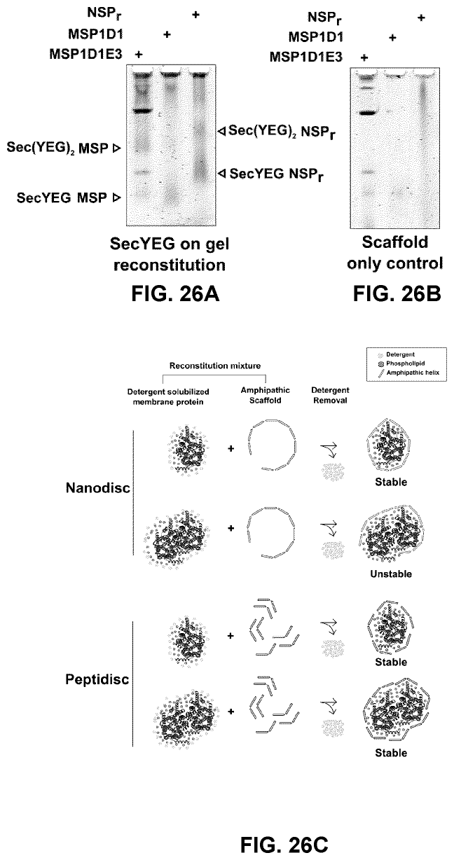

The SecYEG complex (2.5 μg) was incubated with each of the scaffold proteins, NSPᵣ, MSP1D1, MSP1D1E3 (1.25 μg each) in Buffer A+0.02% DDM. Each of the samples was diluted 3-fold in detergent-free Buffer A and immediately analysed by CN-PAGE as shown in FIG. 26A. FIG. 26B shows the results of the same experiment without the addition of SecYEG. FIG. 26C is an illustration of possible reconstitution products of the SecYEGₙ complex into MSP1D1 and NSPᵣ. The results indicate that NSPᵣ is capable of capturing higher-order protein oligomers. The inventors believe that this is because the NSPᵣ peptide can adapt to the size of the protein complex. In contrast, the results suggest that there is an upper limit as to the size of the protein complex that MSP peptide can incorporate and thus be used to purify the protein complex.

### Example 13.0

### Effect of Peptidisc on BRC Stability

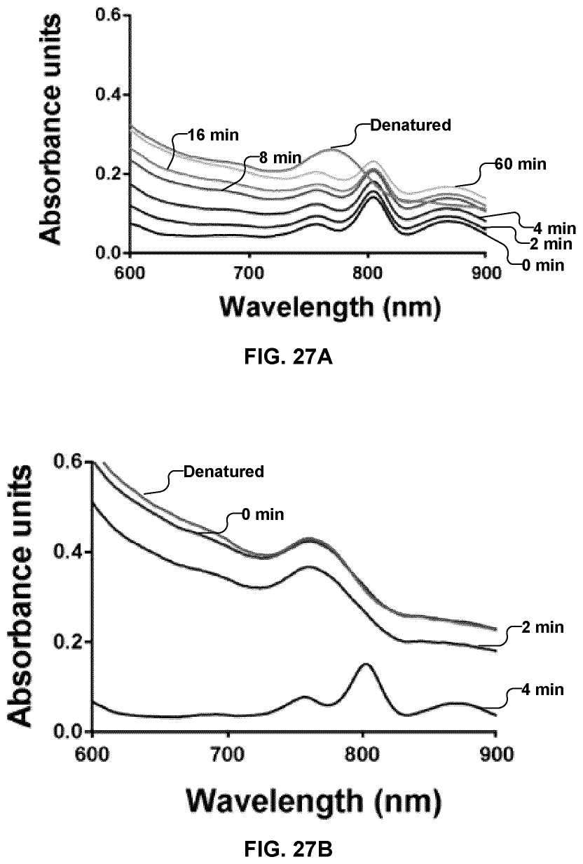

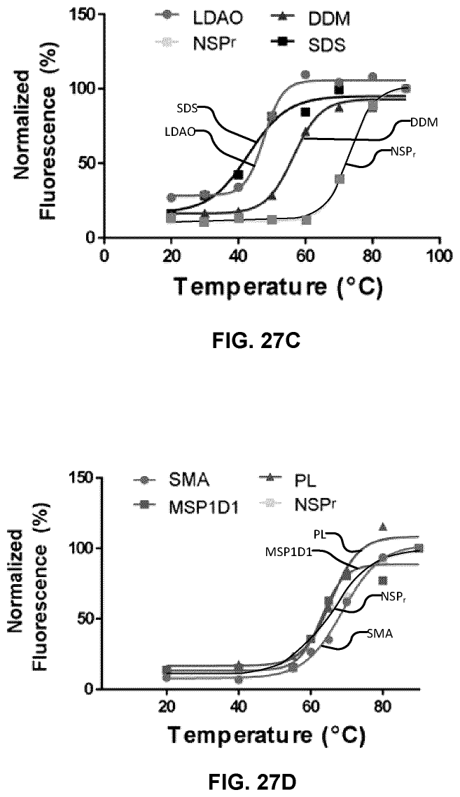

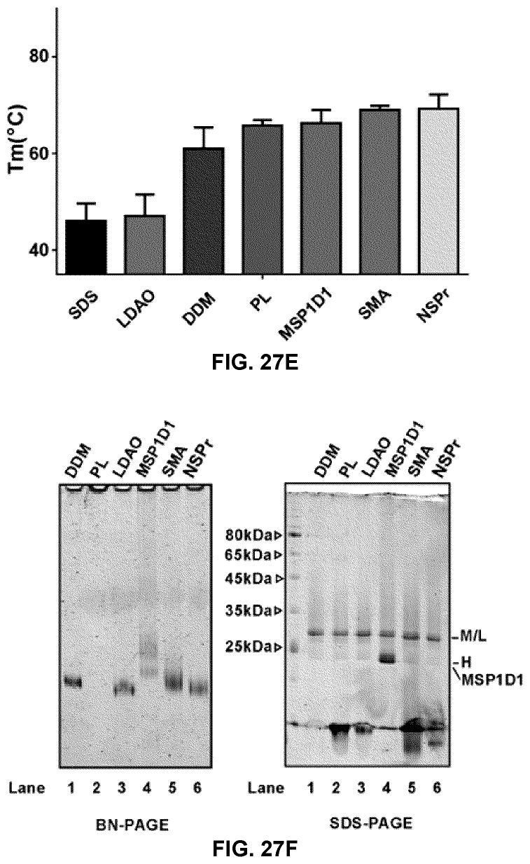

FIG. 27A shows absorbance scans of the BRC complex (1 μM) in peptidisc made using NSPᵣ after incubation at 65° C. for up to 1 hour. Incubation at 90° C. leads to full release of the bacteriochlorophyll pigment. FIG. 27B shows absorbance scans of the BRC (1 μM) after incubation at 65° C. in 0.03% LDAO for up to 4 min. FIG. 27C shows the fluorescence of each of BRC in peptidisc (NSPr), 0.1% LDAO, 0.02% DDM, and 0.1% SDS. The BRC (1 μM) was incubated for 5 minutes at the indicated temperature before fluorescence was measured (700 nm; excitation at 680 nm). FIG. 27D shows the fluorescence of each of BRC reconstituted into MSP1D1 (1:2 BRC:MSP1D1 molar ratio, SMA (0.1%), Proteoliposomes (1:1600:400 BRC:DOPC:DOPG), and peptidiscs. Fluorescence values were normalized to 100% after denaturation for 5 minutes at 90 degrees. Data were fitted using a Boltzmann sigmoidal function to calculate the melting temperature (Tm) as shown in FIG. 27E. FIG. 27F shows the results of analysis of reconstituted BRC fractions on BN-PAGE (left panel) and SDS-PAGE (right panel).

### Example 14.0

### Example of Library Construction Using Model Bacterial Membrane

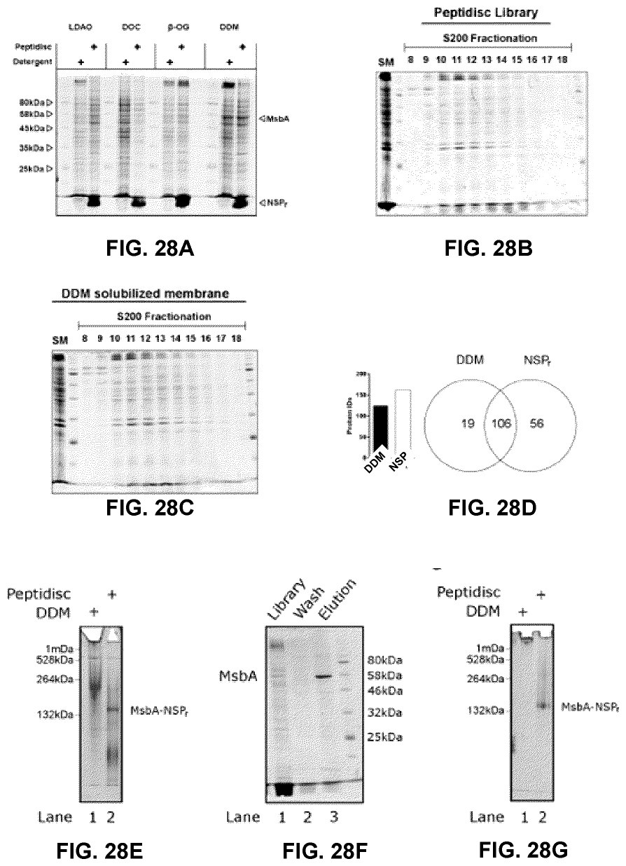

To demonstrate that peptidisc can be used to study in vivo protein-protein interactions, FIG. 28A illustrates the results of a SDS-PAGE analysis of detergent solubilized E. coli crude membrane before and after reconstitution into peptidiscs. The crude membrane preparation was solubilized in either 1% n-dodecyl-beta-maltoside (DDM), 3% β-octyl glucoside (β-OG), 1% sodium deoxycholate (DOC), or 1% lauryldimethylamine-N-oxide (LDAO), followed by reconstitution into peptidiscs by dilution and buffer exchange. FIGS. 28B and 28C show the SEC-fractionation of DDM extract (FIG. 27C) and peptidiscs library prepared from DDM extract (FIG. 28B). A total of 20 fractions were collected, and the fraction containing the highest concentration of protein (fraction 12) analyzed by electrospray mass spectrometry in triplicate. The mass spectrometry data was searched together in Maxquant.

FIG. 28D shows a comparison between the number of proteins identified in fraction #12 of the DDM extract and the peptidiscs library prepared from the DDM extract. A total of 125 proteins and 162 proteins were identified from the detergent and peptidisc samples respectively, with about 85% overlap between the two samples. The detergent sample requires acetone precipitation prior to MS analysis, resulting in a lower protein recovery as compared to the peptidisc sample. These results demonstrate that peptidiscs are well-suited for mass spectrometry analysis of membrane proteomes.

FIGS. 28E to G illustrate results from the purification of MsbA from the peptidisc library. FIG. 28E illustrates the results of CN-PAGE analysis of crude membrane solubilized in DDM (Lane 1) or in peptidiscs (Lane 2). FIG. 28F illustrates the peptidisc library containing overexpressed MsbA (Lane 1) then bound to Ni-NTA beads, washed in Buffer A (Lane 2), and eluted in Buffer A+250 mM imidazole (Lane 3). The samples were analysed by SDS-PAGE. FIG. 28G illustrates the results of a CN-PAGE analysis of MsbA purified from the DDM extract (Lane 1) or purified from the peptidiscs library (Lane 2).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

All references cited in this specification are incorporated by reference herein in their entireties.

1. An amphipathic scaffold peptide having an amino acid sequence having the general formula, from the N-terminus to the C-terminus, AH₁-linker-AH₂, wherein AH₁ and AH₂ each comprise at least one aromatic amino acid residue, and wherein the number of aromatic amino acid residues in AH₁ is greater than or equal to the number of aromatic amino acid residues in AH₂.

2. (canceled)

3. An amphipathic scaffold peptide as defined in claim 1, wherein AH₁ and AH₂ each comprise at least one aromatic amino acid residue, and the scaffold peptide comprises an aromatic amino acid residue at one or more of residue position numbers 1, 13 or 32 from the N-terminus of the peptide, and wherein the aromatic amino acid residue optionally comprises a phenylalanine.

4. (canceled)

5. An amphipathic scaffold peptide as defined in claim 3, wherein a total of at least about eight intra-helix salt bridges are formed between charged amino acids of each of AH₁ and AH₂.

6. An amphipathic scaffold peptide as defined in claim 5, wherein AH₁ and AH₂ each comprise at least two charged amino acid residues that form an intra-helical salt bridge, and one of the at least two charged amino acid residues is located at residue position numbers 4, 6, 10, 15, 23, 25, 29 or 34 from the N-terminus of the peptide, and wherein the one of the at least two charged amino acid residues optionally comprises a positive charge.

7. (canceled)

8. (canceled)

9. An amphipathic scaffold peptide as defined in claim 6, wherein AH₁ and AH₂ each comprise at least two charged amino acid residues that form an intra-helical salt bridge, and one of the at least two charged amino acid residues is located at residue position numbers 3, 7, 11, 22, 26, 30, 37 from the N-terminus of the peptide, and wherein the one of the at least two charged amino acid residues optionally comprises a negative charge.

10.-12. (canceled)

13. The amphipathic scaffold peptide as defined in claim 9, wherein the linker flexibly connects AH₁ and AH₂, and wherein the linker comprises between 1 and 10 amino acid residues and optionally comprises at least one of a proline (P), glycine (G) or alanine (A) amino acid residue.

14. (canceled)

15. The amphipathic scaffold peptide as defined in claim 1, wherein AH₁ and AH₂ each comprise an amino acid sequence having a general formula from the N-terminus to the C-terminus, (Pho)ₐ-(Phi)_{b}-(Pho)_{c}-(Phi)_{d}-(Pho)ₑ-(Phi)_{f}-(Pho)_{g}-(Phi)ₕ-(Pho)ᵢ-(Phi)ⱼ, wherein a is 1-3, b is 2, c is 1, d is 2, e is 2, f is 2, g is 3, h is 1, i is 2 and j is 1, and wherein Pho is any hydrophobic amino acid residue and Phi is any hydrophilic amino acid residue, and wherein a is 2 and wherein one of the hydrophobic amino acid residues in (Pho)_{g} is optionally an aromatic acid residue.

16.-17. (canceled)

18. The amphipathic scaffold peptide as defined in claim 15, wherein AH₁ and AH₂ each comprise an amino acid sequence having a general formula from the N-terminus to the C-terminus, (Pho)ₐ-Neg-Pos-(Pho)_{b}-Pos-Neg-(Pho)_{c}-Pos-Neg-(Pho)_{d}-Pos-(Pho)ₑ-Neg wherein a is 2, b is 1, c is 2, d is 3, e is 2, and wherein Pho is any hydrophobic amino acid residue, Neg is any negatively charged amino acid residue and Pos is any positively charged amino acid residue, and wherein one of the hydrophobic amino acid residues in (Pho)_{d} optionally comprises an aromatic amino acid residue.

19. (canceled)

20. The amphipathic scaffold peptide as defined in claim 18, wherein AH₁ comprises an amino acid sequence represented by a general formula from the N-terminus to the C-terminus, Aro-Sma-Neg-Pos-Aro-Pos-Neg-Sma-Pho-Pos-Neg-Aro-Aro-Sma-Pos-Aro-Aro-Neg, and AH₂ comprises an amino acid sequence represented by a general formula from the N-terminus to the C-terminus, Sma-Sma-Neg-Pos-Pho-Pos-Neg-Sma-Pho-Pos-Neg-Aro-Aro-Sma-Pos-Pho-Aro-Neg, wherein Neg is any negatively charged amino acid residue, Pos is any positively charged amino acid residue, Aro is any aromatic amino acid residue and Sma is any small hydrophobic amino acid residue.

21. The amphipathic scaffold peptide as defined in claim 20, wherein each of AH₁ and AH₂ comprise an amino acid sequence from N-terminal to C-terminal of (Pho)₂-(Phi)₂-Pho-(Phi)₂-(Pho)₂-(Phi)₂-(Pho)₃-Phi-(Pho)₂-Phi, wherein Pho is any hydrophobic amino acid residue and Phi is any hydrophilic amino acid residue.

22. The amphipathic scaffold peptide as defined in claim 21, wherein the amino acid sequence of the amphipathic scaffold peptide is represented by a general formula from the N-terminus to the C-terminus, Aro-Sma-Neg-Pos-Aro-Pos-Neg-Sma-Pho-Pos-Neg-Aro-Aro-Sma-Pos-Aro-Aro-Neg-Pro-Sma-Sma-Neg-Pos-Pho-Pos-Neg-Sma-Pho-Pos-Neg-Aro-Aro-Sma-Pos-Pho-Aro-Neg, wherein Neg is any negatively charged amino acid residue, Pos is any positively charged amino acid residue, Aro is any aromatic amino acid residue and Sma is any small hydrophobic amino acid residue.

23.-26. (canceled)

27. The amphipathic scaffold peptide as defined in claim 13, wherein the ratio of aromatic amino acid residues in the amino acid sequences of AH₁ and AH₂ is 4:1, 3:1, 3:2, 2:1, 4:3, 4:2, 4:4, 2:2 or 1:1.

28.-41. (canceled)

42. The amphipathic scaffold peptide as defined in claim 1, having the amino acid sequence set forth in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 12, SEQ ID NO: 13, SEQ ID NO: 14, SEQ ID NO: 15, SEQ ID NO: 16, SEQ ID NO: 17, SE ID NO: 18, SEQ ID NO: 19, SEQ ID NO: 20, SEQ ID NO: 21, SEQ ID NO: 22, SEQ ID NO: 23, SEQ ID NO:24, SEQ ID NO: 25, SEQ ID NO: 26, SEQ ID NO: 27, SEQ ID NO: 28, or SEQ ID NO: 29, or an amino acid sequence having at least 80% sequence similarity to SEQ ID NO: 2.

43.-44. (canceled)

45. The amphipathic scaffold peptide as defined in claim 27, wherein the amphipathic scaffold peptide has a solubility of at least 5 mg/mL in water.

46. The amphipathic scaffold peptide as defined in claim 45, wherein the number of inter-helix salt bridges formed between AH₁ and AH₂ is greater than about 40.

47.-71. (canceled)

72. The amphipathic scaffold peptide as defined in claim 46, wherein the total number of amino acid residues in the amphipathic scaffold peptide is in a range of about 30 to 45.

73.-82. (canceled)

83. A nanoscale particle comprising:
an amphipathic scaffold peptide as defined in claim 1; and
a protein having at least one hydrophobic region,
wherein, in an aqueous solution, the amphipathic scaffold peptide is self-assembled in a tilted orientation at the at least one hydrophobic region of the protein, and wherein one or more hydrophobic amino acid residues in the amphipathic scaffold peptide interacts with one or more hydrophobic amino acid residues in the at least one hydrophobic region of the protein in aqueous solution.

84.-85. (canceled)

86. The nanoscale particle as defined in claim 83, wherein the protein is a membrane protein, wherein the membrane protein is optionally one of an alpha-helical membrane protein, a beta-barrel membrane protein, a transmembrane protein (TMS), a monomeric membrane protein or an oligomeric membrane protein.

87.-88. (canceled)

89. A method of stabilizing a membrane protein, comprising the steps of:
obtaining the membrane protein by isolating the membrane protein from a membrane by adding a solubilizing agent to a solution containing the membrane and the membrane protein; and
combining the membrane protein with one or more of an amphipathic scaffold peptide as defined in claim 1, resulting in the amphipathic scaffold peptide self-assembling around one or more hydrophobic regions of the membrane protein to yield a nanoscale particle.

90.-92. (canceled)

93. The method according to claim 89, wherein the step of combining the membrane protein with the amphipathic scaffold peptide is performed using any one of size-exclusion chromatography, gel electrophoresis, affinity chromatography, density gradient centrifugation, or by removing unbound/non-incorporated lipids and/or peptides and/or detergent by: dialysis, detergent-binding biobead separation, magnetic bead separation, concentrators, or membrane filtration.

94.-104. (canceled)
